(19) United States
(12) Patent Application Publication
(10) Pub. No.: US 2020/0097910 A1
(43) Pub. Date: Mar. 26, 2020

(54) A SYSTEM FOR GENERATING A RECORD OF COMMUNITY-BASED PATIENT CARE

(71) Applicant: SENSORY TECHNOLOGIES OF CANADA INC., London (CA)

(72) Inventors: Andrew Krasnov, London, Ontario (CA); Michael Andrew Paget, London, Ontario (CA)

(21) Appl. No.: 16/621,527

(22) PCT Filed: Jun. 12, 2018
(86) PCT No.: PCT/CA2018/050698

§ 371 Date: Dec. 11, 2019

Related U.S. Application Data
(60) Provisional application No. 62/518,544, filed on Jun. 12, 2017.

(51) Int. Cl. G06Q 10/10; G06F 16/25; G16H 10/60; G16H 20/00; G16H 80/00; G16H 40/67; G16H 40/20

(52) U.S. Cl. CPC G06Q 10/101; G06F 16/252; G16H 10/60; G06F 3/0484; G16H 80/00; G16H 40/67; G16H 40/20; G16H 20/00

(57) ABSTRACT

A system for enabling the delivery of healthcare services is provided. The system has a server with a database that stores an electronic community care record (ECCR). The system also has a directing workstation. The directing workstation has a user interface for allowing a licensed healthcare professional to access the ECCR. The system further has a mobile device. The mobile device has a user interface for allowing a healthcare assistant to access the ECCR. The ECCR has an entity history index that contains data corresponding to an action performed on the user interface of the directing workstation and the mobile device.

### COPYRIGHT AND TRADEMARK NOTICE

A portion of the disclosure of this patent document contains material, which is subject to copyright protection. The copyright owner has no objection to the facsimile reproduction of the patent document or the patent disclosure, as it appears in the Patent and Trademark Office patent file or records, but otherwise reserves all copyright rights whatsoever. Trademarks are the property of their respective owners.

### BACKGROUND

Health care costs are continually escalating along with the number of individuals requiring extended care, placing a number of strains on the ability of health care providers to deliver appropriate expertise in a cost effective manner. For example, in 2016, it has been estimated that nearly 44 million people world-wide have Alzheimer's or a related dementia, with a global cost of $605 billion; in the U.S. alone, 5.3 million people have Alzheimer's, with the number expected to raise to 16 million by 2050. Another example of patients requiring extended care are those having moderate to severe chronic obstructive pulmonary disease (COPD), which is estimated to range between 13-27 million in the U.S.

There are many reasons why it is desirable for a patient to be able to receive extended health care services in a non-hospital location, such as their home, long-term care facility or other location. These reasons include cost efficiencies, better patient outcomes, decreased risk of exposure to hospital “superbugs,” etc.

There are a number of organizations involved in the provision of medical care to a patient located in the community: primary care providers, therapists, payors, managers, auditors, etc. Hospitals may be involved as a patient may have started receiving care within a hospital, following some kind of critical event initiating the course of care (e.g., stroke, heart attack, etc.) or may require recurring visits due to a degenerative medical condition (e.g., COPD, Alzheimer's, cancer, etc.).

Each organization directly or indirectly involved in providing care to a patient tends to have their own records, such that patient data is stored piecemeal within each of the organizations. This system helps to overcome this limitation of health care being provided to patients in non-hospital locations, by generating a centralized repository of data pertaining to the provision of patient care in addition to the patient's personal health information (PHI).

There is also extensive variation in the kind of data that is collected by each individual/organization involved, which results in inconsistent patient data in the records as well as time-consuming challenges to cost attribution procedures. This system helps to overcome these issues.

The economics of health care dictate the need to extend expertise as much as possible. Responsive to this need of extending nursing care a distributed health care system was generated to provide a means for a nurse to remotely monitor the health of a patient located in an environment outside of a hospital, such as a home, long term care facility, hospice, etc. An embodiment of this system is presented in US 2012/0290313, which describes a system for distributed health care that uses personal support workers (PSW) and registered, trained medical personnel. Each PSW is equipped with a mobile computing device that is capable of communicating with a main computer. Each registered medical personnel is equipped with a computing device (a monitoring computer) that is capable of communicating with a main computer. At times during a PSW's shift at a patient location, the PSW inputs data to a number of forms on the mobile computing device, each form being related to the patient's physical appearance, medical condition, medication taken or given, and physical parameters, or other actions taken. The data inputted are then transmitted to the main computer, which processes, stores, and archives the data. After processing, the data is reviewed by the registered medical personnel. If the data indicates that actions need to be taken, the medical personnel can issue instructions to the PSW.

In order to accomplish the partitioning of tasks between a licensed clinician (located in their house or a health care organization, for e.g.) and a trained technician located in the house of a patient the system (hardware, software and processing/workflows) was developed in a unique (stringent) manner. Remotely supervising and directing a technician constitutes a quantum jump from supervising a technician where both are located in a hospital or other institutional setting. This system and processes provide the structure required by the complex regulatory framework governing patient care, enabling the technician to legally operate under the license of the delegating nurse.

In order to accomplish this, a system has to meet the requirements of the local regulatory environment, patient data privacy concerns, amongst other issues in the field of healthcare and patient data collection.

Described from a legal perspective (using the Ontario, Canada regulatory framework as an example), in essence, the foundational platform of the system constitutes an “authorizing mechanism,” which enables the transference of the nurse's authority to perform “controlled acts” to a non-regulated, appropriately skilled technician. The forms presented on the user interface of the technician's mobile device create “orders” through which the nurse (the delegator) directs the technician (the delegatee) to perform controlled acts on their behalf. In Ontario, there are 10 specific requirements, which must be satisfied for this transference of authority to occur. Requirement 10 states that the delegating nurse shall:

a) ensure that a written record of the particulars of the delegation is available in the place where the controlled act is to be performed, before it is performed, or

b) ensure that a written record of the particulars of the delegation, or a copy of the record, is placed in the client record at the time the delegation takes place or within a reasonable period of time afterwards, or

c) record particulars of the delegation in the client record either at the time the delegation takes place or within a reasonable period of time afterwards.

Thus, any system enabling the transference of a licensed health care professional's licensed to a non-licensed individual to perform “controlled acts” must minimally meet these kinds of local regulatory requirements. Since the regulations require written records of the particulars of the delegation, which would vary from patient type to patient type (e.g., COPD vs. stroke vs. Alzheimer's, etc), any system must be easily adaptable in order to be able to generate the particulars of each delegated event.

### The Need for Increasingly Detailed Records

Another trend in health care is the increasing number of agencies and organizations responsible for providing care to a patient, either directly in terms of a health care providing organization, or indirectly in terms of payor organization(s), for example. There are also organizations or departments within traditional organizations responsible for monitoring the performance of the health care providers. In some locales, a number of different agencies and organizations are responsible a patient and their health care, so need to be informed of the patient's care plan, how effectively the care plan is being delivered, how the patient is doing, whether more, less or alternate services are required, etc.

Many of these organizations require appropriate (i.e., non-personal), accurate and detailed information about a patient and the care they are being provided with, usually not including the patient's personal health data, but what could be considered the particulars or meta-data involving the provision of care. An example of particulars or meta-data could be the amount of time clinicians spend discussing a patient, the timing of patient interventions and/or clinician activities surrounding the patient, when and how often patient data is collected, etc. A lot of this meta-data is not captured by traditional patient care documentation protocols and is therefore missing from a traditional patient record. Thus, a need remains for the ability to collect and document in the patient record, the particularities involved in providing care to a patient.

There are various aspects to a patient health record, generally divisible into two aspects—those pertaining to the information, which provides a foundation to the health data of the patient, such as for example, their age, address, family history of disease/health, insurance providers, list of current medications, etc. This kind of information can be collected by different kinds of clinicians and/or their support staff and could be considered “mandatory (compulsory) patient information,” depending on the legal and business requirements of the jurisdiction and organization(s) caring for a patient.

There are also aspects of a patient medical record that pertain to the health status of the patient, such as patient data (e.g., vital signs, psychological status, etc.), which can generally only be collected by an appropriately licensed clinician or a specially trained technician working under the direct supervision of a licensed clinician.

A doctor typically collects patient health data during patient visits either for a routine periodic visit or for some reason of medical concern and documents their observations and findings. Patient records are also generated in a hospital, once a patient is admitted. Either way, the records would be generated on an “as needed basis,” usually with the minimal documentation, subject to the judgment calls of busy professionals.

In most medical settings, such as a hospital or a long-term care facility, the clinicians do not generally continuously document many of the various health indicators of a patient in an ongoing manner. In general, a clinician (doctor or nurse) is very busy caring for a number of patients and juggling the various tasks and responsibilities required for those patients, such that they are not able to focus solely on patient data collection and documentation. Rather the clinicians tend to observe the patients and then make judgment calls when the situation has changed significantly and new patient data most relevant to the change in the patient's condition needs to be collected and documented. In these kinds of scenarios, the patient data collection and documentation tends to be more in response to a situation.

Clinicians are licensed to make judgment calls all the time, including what data to collect and record. Deciding what to record, when and how much form part of the discretion afforded under a license. These aspects of a patient medical record could be considered discretionary patient data.

For these reasons, during a patient examination, a busy clinician will generally conduct an assessment and make judgment calls as to what patient data they will collect and record. Apart from the basics (e.g., blood pressure, heart rate, listening to the heart and lungs with a stethoscope, etc.), clinicians do not generally collect standardized data sets of patient data. Nor do they follow a standardized workflow of patient data collection procedures. In general, patient data is collected and documented in response to a situation, such as the worsening of a patient's symptoms and/or condition. The comprehensiveness of the patient data set and the timing of the data collection could be considered to be responsive to a situation.

These factors generally give rise to patient records that can be inconsistent in their collection and documentation of patient data, especially if a patient is being cared for in the community and not a hospital. Even a patient being cared for in a long-term care facility is likely being supervised (observed) and data being collected only when considered important to do (weighed against the other tasks the clinician is required to accomplish).

One attempt to collect more continuous patient data has led to the proliferation of Remote Patient Monitoring (RPM) devices were introduced to provide continuous data collection with—“beep alerts”, to indicate a problem has occurred. In practice, however busy, overtasked humans simply develop “alert desensitation,” ignore the beeps and in some cases turn off the alarm. In addition, these kinds of RPM-alarm systems only report when a physiological signal has exceeded a range of “normal.” They can not provide any context to the changes in the physological data, nor provide warning based on the trends in the data.

Some of the most recent advances to generating patient records have come about by computer software, for example computerized transcription services for clinician's verbal observations. However, these systems have generally been developed to mirror and support the usual practices involved in delivering patient care. In some jurisdictions, this kind of software is heavily focused around billing practices.

For these reasons, a need exists for a system that is able to enable and facilitate the ability to provide delegated/directed health care, generate extensive documentation regarding the details of the provision of health care including patient data, facilitate cost attribution and billing procedures, as well as other requirements of community health care.

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also be inventions.

### SUMMARY

The system comprises an authorizing mechanism platform (server/database/database management software, workstations, mobile devices configured by webpages) enabling the delivery of health care services to a non-hospital-based patient and the generation of an Electronic Community Care Record (ECCR), which documents the details of how the health care was provided in addition to extensive patient data. The system is also configured to enable cost attribution of the activities events giving rise to the delivery of health care to a patient and in some embodiments in an authorizing manner (i.e., the provision of care restricted to pre-approved costing).

One aspect of the system comprises an Entity History Index within a relational database, where metadata pertaining to various aspects of the delivery of health care to a patient are recorded. The comprehensiveness of the data recorded in the Entity History Index supports the generation of an ECCR, which contains the detailed chronological history of the provision of the care to a patient in addition to extensive and comprehensive data sets of patient data. The configuration of the Entity History Index also enables health care information to be provided, which excludes a patient's personal health information (PHI).

One aspect of this system comprises web pages displayed on the user interface of mobile devices as “forms,” wherein each web page/form comprises form metadata. The form history metadata chronicles the legal approval of each web page, enabling the provision of remote health care by an individual working under the delegation and/or direction of a clinician.

The form metadata also enables the chronological tracking of the delivery of health care and it's reporting in the ECCR of each patient, in a manner that separates the use of the form from the confidential patient personal health information (PHI). The form metadata also facilitates trafficking and notification of each form's use.

Another aspect of the system comprises PatientServiceIds, which are attached to each session wherein an individual in an authorized role enters into a patient record to perform some service pertaining to the provision of health care to a patient. In one embodiment, PatientServiceIds are used by the system to track events related to the opening of a patient record and are associated by the system to codes provided by third parties such as a “Billing Reference Number” (BRN). The system records the time and duration of each session. PatientServiceIds can be used to facilitate cost attribution processes, conducting analytics on the distribution and delivery of health care resources, etc.

This system enables health care providers (clinicians, therapists, technicians/assistants, payors, managers, etc.) to work in integrated teamwork, because their communications are all connected through a patient's ECCR, which facilitates everyone seeing the same documentation to the degree they have authorized access to various sections of the record, in addition to the system documenting the interactions.

### FIGURES

FIG. 1 depicts an example relationship of some of the parties of the system according to one embodiment of the system.

FIG. 2A-2D depict example forms according to one embodiment of the system as presented on a mobile device.

FIG. 3A and FIG. 3B depict an example user interface (UI) according to one embodiment of the system that is provided to the supervising therapist (a speech language pathologist) on their workstation.

FIG. 4A and FIG. 4B illustrate two exemplary main exemplary navigational menus. FIG. 4A presents a main system menu for the sub-menus: Inbox, Patient, Dashboards, Admin, Resources, and Contacts. FIG. 4B presents a main menu accessible for each patient with the sub-menus: Info, Care Team, Activity, Notes, Dash, Forms, Care Team, Clinical History, Shift.

FIG. 5A and FIG. 5C show partial views of the user interface of the Directing Clinician Workstation according to one embodiment of the system. FIG. 5B shows various indicators that can be displayed next to a patient's name, according to one embodiment of the system. FIG. 5D illustrates how the Urgent Section of the shift details displays un-reviewed Alert Events, for example, a Risk Event reported by other Care Team Members.

FIG. 6, according to one embodiment of the system, illustrates the workflow of communication between a Directing Nurse (DN) and a technician (PSW), using “snippets” of user interfaces as displayed on the mobile of the technician 520 and the Directing Workstation of the DRN (directing registered nurse).

FIG. 7 is an entity relationship diagram, according to one embodiment of the system, illustrating some of the conceptual relationships between the Entity History Index Table and a number of other tables within the relational database.

FIG. 8 is an entity relationship diagram illustrating the conceptual relationship between the Entity History Index and the tables comprising patient data demonstrating one embodiment of the system.

FIG. 9 illustrates a user interface on a Directing Clinician Workstation according to one embodiment of the system, wherein a clinician has selected the Clinical History tab and selected a number of History Details for review.

FIGS. 10A and 10B demonstrate one embodiment of the system. FIG. 10A presents a partial view of a user interface on a Directing Clinician Workstation, wherein the Clinician has selected the Meds tab. FIG. 10B presents a partial view of a user interface on a Directing Clinician Workstation, wherein the Clinician has filtered the results by Date.

FIGS. 11A and 11B demonstrate one embodiment of the system. FIG. 11A presents a partial view of a user interface on a Directing Clinician Workstation, wherein the clinician has selected the Activity tab to reveal the Activity History screen. FIG. 12B presents a partial view of a user interface on a Directing Clinician Workstation, wherein the clinician has selected the Notes tab to reveal the Notes screen.

FIGS. 12A and 12B demonstrate one embodiment of the system. FIG. 12A presents a partial view of a Main Menu on the user interface on a Directing Clinician Workstation, wherein the clinician has selected the Dashboard tab to reveal a dropdown menu, from which they selected the Clinical Record Dashboard, as illustrated in FIG. 12B.

FIGS. 13A-E illustrate how notifications are presented on the user interface of a Directing Workstation.

FIGS. 14A-C show three examples of user interfaces allowing a clinician to select an instruction to be sent to a technician.

FIG. 15 depicts an example work flow diagram of the communications between a Directing Clinician and a Technician regarding an instruction and the manner in which the system updates the status of the instruction within the data stores of the system, according to one embodiment of the system.

FIG. 16 illustrates data workflow between some members of a Care Team and members of a third party, according to one embodiment of the system.

FIGS. 17A and 17B illustrate one embodiment of the roles and permissions aspects of the system for the members of a patient's Care Team and some third-party members.

FIG. 18 illustrates a user interface showing how the system provides a list of all the active members of a patient's Care Team for the user to select.

FIG. 19 depicts an example work flow diagram of a chat room functionality according to one embodiment of the system.

FIG. 20 shows an example workflow for a consultation, including the Requestor, the System and the Consultant according to one embodiment of the system.

FIG. 21 describes, according to one embodiment of the system, an example work flow diagram for conducting a multi-party meeting, including the Meeting Master, the System and the Online Meeting Attendee.

FIG. 22 is an entity relationship diagram, according to one embodiment of the system, illustrating some of the conceptual relationships between the Entity History Index Table and a number of other tables within the relational database pertaining to user authentication, user access, and access log among other aspects such as BRN.

FIG. 23 presents a partial user interface on a Directing Clinician Workstation according to one embodiment of the system illustrating a situation in which a nurse is arranging for a technician to visit a patient with Chronic Obstructive Pulmonary Disease (COPD) and the role of BRN/PatientServiceIds. The partial UI represents a form a Nurse may need to complete prior to accessing a Patient Record.

FIG. 24 illustrates one embodiment for some of the ways that a BRN/PatientServiceId might be used in a system.

FIG. 25 shows a user interface wherein the Forms Tab has been selected, demonstrating how the Forms available correspond to a diagnosis, in this case COPD.

FIG. 26 shows a block diagram of an example embodiment is depicted, wherein the dynamic form system includes a form service, a data service, a database, and a user interface.

FIG. 27 presents a block diagram of an example embodiment for creating form templates, wherein the user interface is configured to communicate directly with the form service so that a user may define form templates.

FIG. 28 is a block diagram of an example embodiment depicting the dynamic form system and the underlying data formats/markup languages used in an example dynamic form system.

FIG. 29 a block diagram of an example workflow and data flow diagram for an input/output dynamic form template is provided according to one embodiment of the system.

FIG. 30 block diagram of an example workflow and data flow diagram for a read-only dynamic form template is provided according to one embodiment of the system.

### DETAILED DESCRIPTION

The following detailed description is merely exemplary and is not intended to limit the described embodiments or the application and uses of the described embodiments. As used, the word “exemplary” or “illustrative” means “serving as an example, instance, or illustration.” Any implementation described as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure. The scope of the invention is defined by the claims. For the description, the terms “upper,” “lower,” “left,” “rear,” “right,” “front,” “vertical,” “horizontal,” and derivatives thereof shall relate to the examples as oriented in the drawings. There is no intention to be bound by any expressed or implied theory in the preceding Technical Field, Background, Summary or the following detailed description. It is also to be understood that the devices and processes illustrated in the attached drawings, and described in the following specification, are exemplary embodiments (examples), aspects and/or concepts defined in the appended claims. Hence, dimensions and other physical characteristics relating to the embodiments disclosed are not to be considered as limiting, unless the claims expressly state otherwise. It is understood that the phrase “at least one” is equivalent to “a”. The aspects (examples, alterations, modifications, options, variations, embodiments and any equivalent thereof) are described regarding the drawings. It should be understood that the invention is limited to the subject matter provided by the claims, and that the invention is not limited to the particular aspects depicted and described.

The system comprises a relational database, relational database management system, and applications configured to enable a clinician to remotely monitor an appropriately skilled technician to care for a patient in a non-hospital location, such as the patient's home. The system uses web pages (forms) to collect and store data in the various tables within the relational database, including patient data (e.g., vital signs, medications taken, symptoms, performance in therapy sessions, etc.) and events pertaining to the delivery of care (e.g., the timing of a consultation, an instruction sent to a technician, a change in the members of the care team, an alert sent to a clinician, etc.). In one embodiment, the system comprises means to link the delivery of medical care to the payor of the service. The system can render various types of reports pertaining to the delivery of the care (data and events) thereby generating comprehensive electronic community care records (ECCR).

### The Basic Design of the System

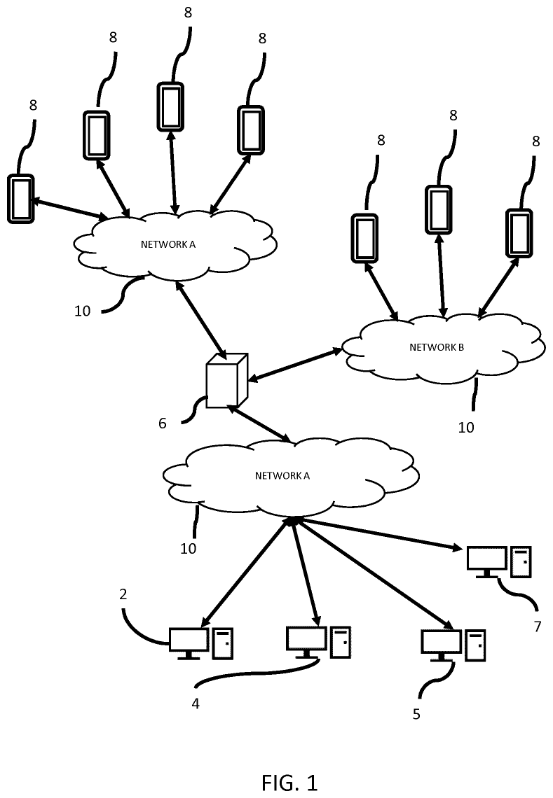

FIG. 1 illustrates one embodiment of a configuration of the system used to deliver care outside of a hospital. For example, the patient location may be the patient's home, an outpatient facility, a nursing home, and other non-hospital or non-clinical facility.

One skilled in the art would appreciate that there are not two separate internets in reality (ie., Network A and Network B) and that the separate “clouds” have been used to denote the plurality of mobile devices/mobile users/patients overseen by each directing clinician. For example, Directing Clinician A oversees the mobile device users associated with “Network A” and Directing Therapist B oversees the mobile device users associated with “Network B.”

FIG. 1 illustrates that the clinicians, therapists and their workstations may be located in many different locations, as long as they have a secure internet connection. For example, all of the clinicians can be located in their homes, or some of them might be located in a health care facility. FIG. 1 also shows how one or more administrators can also join into the system and can be located wherever they have access to a secure internet connection.

In particular, FIG. 1 depicts the relationship between a Directing Clinician (on a Directing Clinician Workstation 2, in this case a nurse) and a plurality of mobile users, each on a mobile device 8 (for example, a technician) providing health care to a patient at a remote location. A clinician, on a Directing Clinician Workstation 2, is tasked with managing a unique set of selected mobile users, each on a mobile devices 8 to provide health care to a patient located in a non-hospital facility. All communications between the Directing Clinician Workstation 2 and the mobile devices 8 is managed and facilitated by the Server/Database 6. It will be understood that any method of networking can be used to communicatively connect the Directing Clinician Workstation 2, the mobile devices 8, and the Server/Database 6 to each other. Examples of networking include, but are not limited to, a VPN, cellular, a LAN, WiFi, etc.

In this embodiment depicted in FIG. 1, the system also includes a Directing Therapist Workstation 5 and a plurality of mobile computing devices 8, notionally connected through Network B 10 through Server/Database 6. Communications between the mobile computing devices and the directing clinician workstations pass through the Server/Database 6. This allows the Server/Database 6 to intercept, facilitate, process, archive, store and/or relay communications between the clinicians or therapists and the mobile device users. Communications between the Server/Database 6 and any of the clinician or therapist workstations also pass through a suitable and secure data network. Networks and may be of the same type of data communications network or they may be dissimilar types of communications networks. The networks may include the Internet, a dedicated local area network (LAN), a virtual private network (VPN), or any other data network that may be used to communicate and transfer data between two data processing devices. In another example the server 6 may be implemented in a cloud computing environment. For instance, the server 6 may be implemented on one or more virtual private servers in a cloud-computing environment such as AMAZON EC2, GOOGLE COMPUTE ENGINE or other such system.

The term, “directing user,” will refer to the clinician or licensed professional using a Directing Clinician Workstation. Some examples of a directing user can be a nurse, physician, therapist, etc. The user on the mobile device can be referred to as the “directed user.” Some examples can be a technician, a therapist assistant, a lower-band nurse (than the band of the directing nurse), and in some cases can be a family member or the patient themselves.

The Server/Database 6 may include one or more data stores for storing data and metadata. In some examples the Server/Database 6 includes separate databases for storing clinical, patient, and caregiver data. In some examples the data from one database may be replicated or copied to another database so that the other database contains a subset of data from the first database, such as anonymized data. In another example, the data in each of the databases may also be encrypted.

In some examples the Directing Clinician Workstation 2 and/or the Directing Therapist Workstation 5 can be a dedicated personal computer, including a laptop, with suitable hardware for communicating with the network or the Server/Database 6. The Mobile Devices 8 can be any suitable mobile computing device that allows users to access the network, display and receive input into preset forms, and which will allow communications with the directing clinician workstation 2, through the server 6. Smart phones, tablet computers, laptops, and any other such device can be used as one of the mobile computing devices. In some embodiments the mobile computing devices have touch screen capabilities.

In one embodiment, mobile user may be a nursing assistant or non-regulated person who attends to a patient for a specific shift (e.g., a 6, 8 or 12 hour shift) during which the mobile user provides care to the patient under the management and supervision of the remote clinician or therapist. During that shift, the mobile user fills out the requested or required forms on the mobile device 8 for the specific patient. The forms have entries for the various physical parameters of each patient that are appropriate for the patient's condition and their surroundings.

### The Data Collection Workflow User Interfaces (UIs)

The system is configured to present a series of user interfaces (UIs) on a mobile device that direct the mobile user to collect and enter specific data for a patient. These UIs present detailed workflows for data collection, which have been specifically designed to meet the requirements of a condition for which a patient is being monitored and/or treated.

This system is so precise in its methods of data collection and documentation that a nurse's license can be extended to a technician caring for a patient in the patient's home. Most medical systems do not need to use data collection workflows, because the clinicians are authorized to make their own judgments around what data is required to be collected, documented, and when. This system is designed to determine the patterns of these data collecting decisions for each kind of patient condition (e.g., chronic pediatric, vs. Alzheimer's, vs. terminally ill palliative), and create data collection work flows reflecting them, which is encapsulated in a UI, (a “form”) or a series of UIs.

In embodiments where a mobile user (e.g., a technician) works a shift, at the beginning of each shift, the mobile user takes readings of the patient's physical parameters (e.g., the patient's mental state and vital signs). These readings are then transmitted to the monitoring computer where the readings are provided to the clinician on the UI of their workstation.

Multiple categories of readings for the patient are also taken. Exemplary readings can be related to the patient's vital signs, neurology, respiratory system, cardiovascular system, skin integrity, and gastrointestinal system, etc. are taken by the mobile user and the patient data is sent to the monitoring computer. This data is then reviewed by the clinician at the directing clinician workstation (and possibly observed by another clinician) to ensure that the results are within acceptable parameters

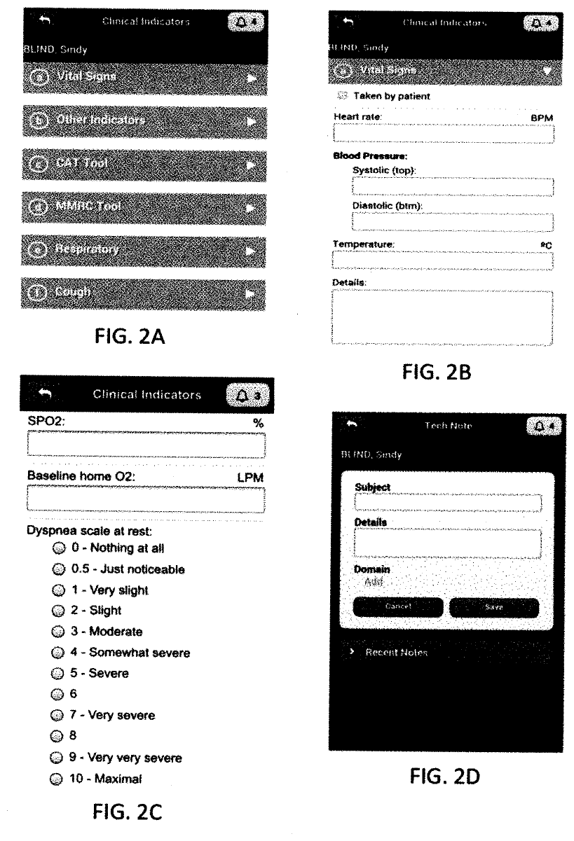

FIGS. 2A-2D present examples of the kinds of UI data collection workflows presented to the mobile user, such as a technician to collect and enter patient data.

Referring now to FIG. 2A, a clinical indicator user interface is depicted on the mobile device 8. This screen presents a series of buttons corresponding to various clinical indicators that a mobile user should enter. Pressing a button will bring up a corresponding data collection workflow user interface for the clinical indicator described on the button.

By way of example, FIG. 2B depicts a vital signs data collection workflow UI. In this example, vital signs such as blood pressure, heart rate, temperature, and details can be recorded. Other vital sign indicators, such as, but not limited to, pallor, reflexes, and pupil sensitivity can be captured without departing from the scope of this disclosure.

Referring now to FIG. 2C, another clinical indicator form is configured to allow a user to enter data related to SPO2, baseline heme O2, and the Dyspnea scale at rest.

Referring now to FIG. 2D, a note form is depicted on the mobile device 8. In this example the note form is configured to allow a user to enter additional notes for observations that may not have been captured in the forms.

Once the mobile user completes these forms (UI's), the data is then transmitted to the directing clinician workstation 2 by way of the Server/Database 6. The data is displayed for review by the clinician on a dashboard display on the directing clinician workstation 2, and may also be presented on the workstation of other clinicians who have selected to observe this mobile user and their patient.

The Server/Database 6 is also configured to store, analyze, and/or process the data and metadata submitted through the forms. The Server/Database 6 is also configured to capture, analyze, and/or process any data from the directing clinician workstation 2 or the observing clinician workstation 4. Data can include, but is not limited to, patient data and any data input in the forms. Metadata can include, but is not limited to, the time the data was entered, the IP address of the mobile device 8, the user of the mobile device, the time it takes to respond to a request from the mobile device, the length of any conversations between the user of the mobile device 8 and the clinician, and/or a recording of any conversations between the user of the mobile device 8 and the clinician. The metadata may also be associated with specific patient data/identifiable patient data so that a more complete patient record/clinical record/patient history, etc. can be compiled for that patient. It should be noted that the data collected, the presentation of the UI (form), and the layouts of the UI (form) can change depending on the data to be collected and the types of patients being cared for. Furthermore, the number of UIs (forms) and order of UIs (forms) can be changed without departing from the scope of this disclosure.

### Therapeutic Service Providers

Some patients located in non-hospital facilities, such as their homes, nursing facility, rehabilitation facility or other extended care facility may benefit from receiving therapeutic services to assist in their recovery and/or to slow down the progression of a chronic disease or disorder. For example, patients who have had one or more strokes, patients with Alzheimer's Disease, amyotrophic lateral sclerosis (ALS), Parkinson's Disease, some form of paralysis (e.g., from a spinal injury), etc. would benefit from having a therapeutic service provider visit them in their home of other non-hospital location.

For the purposes of this description, the term, therapist, includes mental health counselors, occupational therapists, physical therapists, psychologists, registered dietitians, respiratory therapists, speech language pathologists, and orthopedists, etc. and/or any other such clinician. Additionally included in this group of service providers, although not technically therapists, could be clinicians or other skilled professionals acting in more of an administrative capacity conducting check-ups on how the patient, the caregiver and their course of care is proceeding.

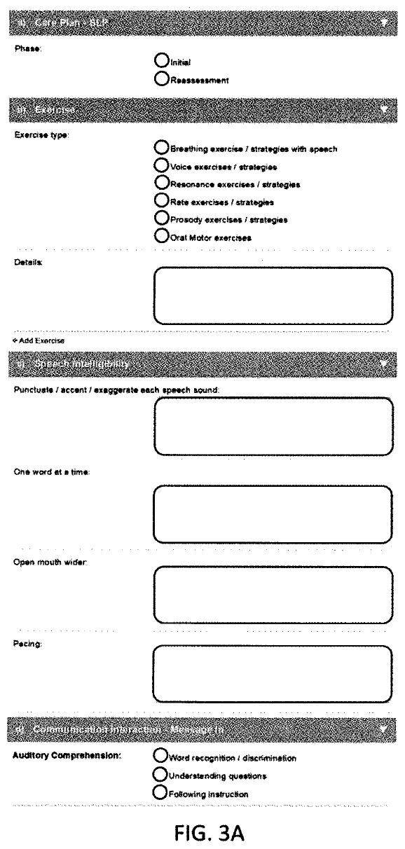

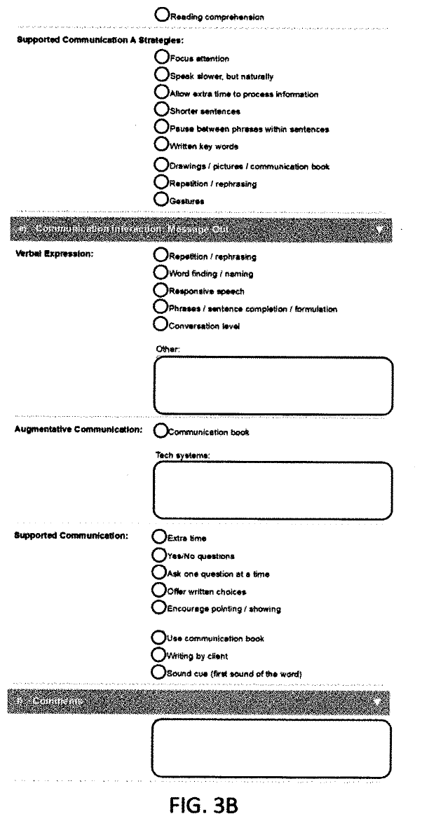

In one embodiment, the system can be used by a therapist, working on a Directing Clinician Workstation and a therapist assistant, using a mobile device. FIGS. 3A and 3B demonstrate an exemplary user interface that the system would generate on the dashboard of a supervising therapist workstation, wherein the therapist is a speech language pathologist (SLP). SLPs (also known as speech language therapists, or speech therapists) are professionals who have training and expertise to work with all ages of patients to evaluate, diagnose and treat a wide range of speech, language, communication, and swallowing disorders.

FIGS. 3A and 3B illustrate the kind of user interface that a supervising SLP might use to create the therapy workflow program for the mobile user to use with the patient. As seen in this example, the supervising SLP selects whether the visit is an initial visit or a reassessment. Then the supervising SLP selects an exercise type from the list provided comprising, for example: breathing exercise/strategies with speech; voice exercises/strategies; resonance exercises/strategies; rate exercises/strategies; prosody exercises/strategies; or oral motor exercises. Once an exercise/strategy is selected, the supervising SLP can enter specific instructions into the section labeled “Details,” for the assistant to follow. The supervising SLP can then select another exercise/strategy by selecting “Add Exercise” and repeating the process of selecting an exercise/strategy and adding specific instructions to go along with the exercise/strategy.

The supervising SLP can then enter instructions pertaining to speech intelligibility by entering instructions into one or more of the sections labeled: “Punctuate/accent/exaggerate each speech sound;” One word at a time;” “Open mouth wider;” or “Pacing.”

The supervising SLP can then select aspects of the patient's capabilities of communication interaction beginning with “message in.” Beginning with auditory comprehension, they can prescribe assessments of the patient's capabilities by selecting: “Word recognition/discrimination;” “Understanding questions;” “Following instruction;” or “Reading comprehension.” Then, addressing the patient's supported Communication A strategies, they can prescribe assessments of the patient's capabilities by selecting: “Focus attention;” “Speak slower, but naturally;” “Allow extra time to process information;” “Shorter sentences;” “Pause between phrases within sentences;” “Written key words;” “Drawings/pictures/communication book;” “Repetition/rephrasing;” or “Gestures.”

The supervising SLP can then select aspects of the patient's capabilities of communication interaction pertaining to “message out,” starting with verbal expression by selecting: “Repetition/rephrasing;” “Word finding/naming;” “Responsive speech;” “Phrases/sentence completion/formulation;” or “Conversation level.” The supervising SLP can enter additional instructions into the section labeled “Other.” The supervising SLP can address the patient's capabilities of augmentative communication by directing the assistant to refer to the communication book to use one or more specific tech systems by selecting “Communication book” and entering specific instructions into the section labeled “Tech systems.” The supervising SLP can then direct the assistant to work with the patient's capabilities of supported communication by selecting: “Extra time;” “Yes/No questions;” “Ask one question at a time;” “Offer written choices;” “Encourage pointing/showing;” “Use communication book;” “Writing by client;” or “Sound cue (first sound of the word).”

Finally, in this example, the supervising SLP can provide any additional instructions to the assistant by entering them into the section labeled “Comments.”

### The Directing Clinician Dashboard

In a traditional situation, a nurse's responsibilities are divided between delivering care, collecting, documenting patient data, ec. Nurses using this system focus primarily on directing the mobile user, observing patient data, and entering commentary into the Clinical Notes, which interprets, integrates and contextualizes the flow of patient data. This kind of contextualized information gathered from such data sets is not present in the traditional patient records.

The Directing Clinician Dashboard, which is presented on the Directing Workstation, receives and displays information that is different from a traditional nurses workstation because of the uniquely structured data that is being collected by the mobile user is different. Thus, the nurses are exerting their discretion based on a different and generally more thorough patient data set.

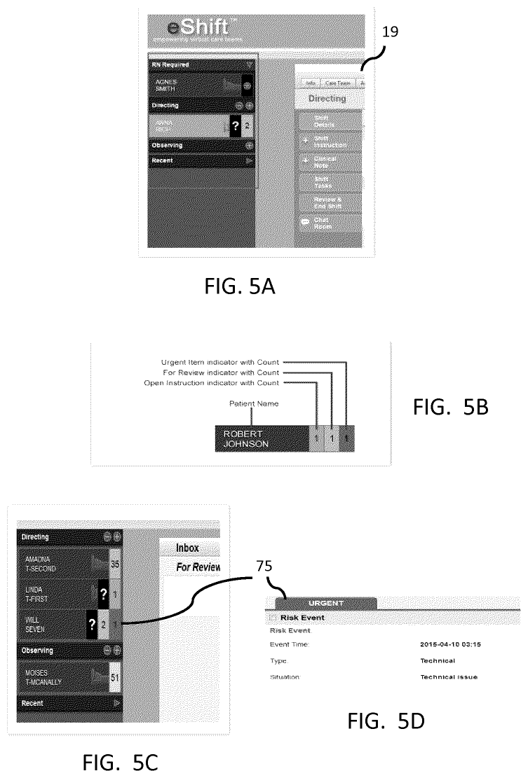

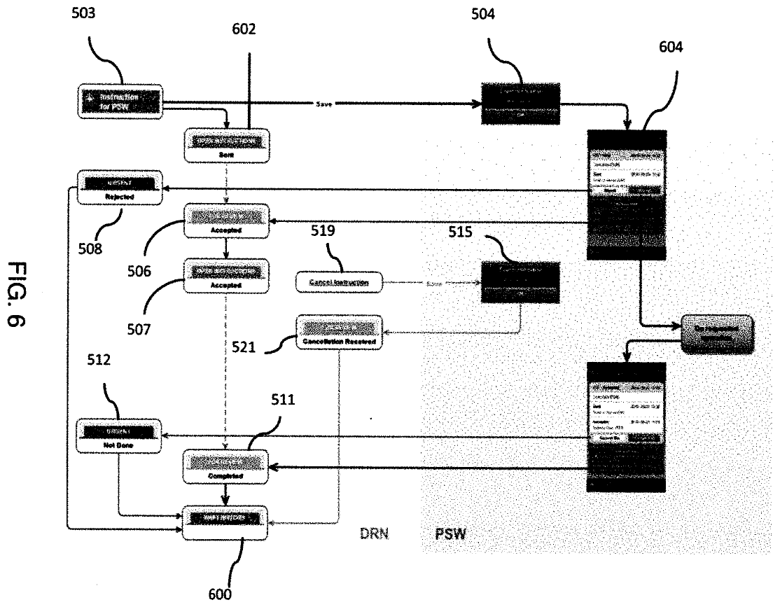

Additionally, the fact that a nurses' dashboard can be divided into views for the mobile users for whom they are the directing clinician, in addition to views for the mobile user/patients for whom they are an observing clinician, along with the permissions accorded to each, is unique. Having the easy ability to “contact-switch” to take on the directing clinician responsibility from another clinician, provides a mechanism by which the nurses can act in teamwork that was not possible without this system. This aspect is illustrated in FIG. 5C, wherein the partial screen shot of the clinician shows that they are directing Amanda T-Second, Linda T-First and Will Seven, while observing Moises T-McNnally.

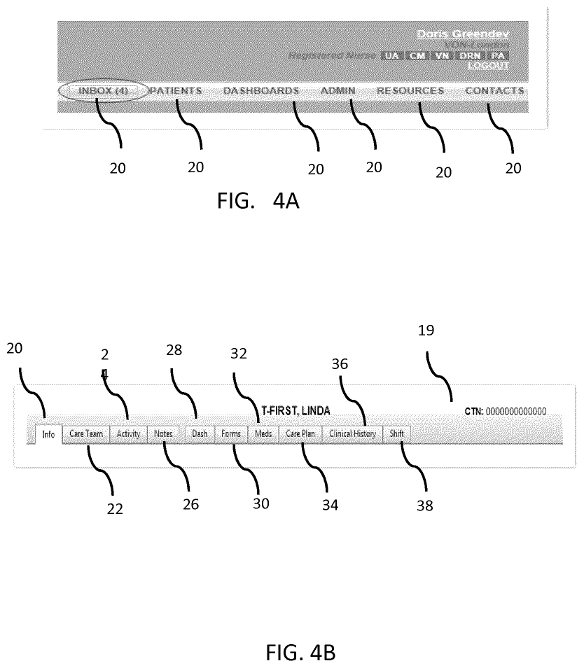

FIG. 4A and FIG. 4B illustrate two exemplary main exemplary navigational menus according to one embodiment of the system. FIG. 4A presents a main system menu for the sub-menus: Inbox, Patient, Dashboards, Admin, Resources, and Contacts. FIG. 4B presents a main menu accessible for each patient with the sub-menus: Info, Care Team, Activity, Notes, Dash, Forms, Care Team, Clinical History, Shift.

Also part of the dashboard for the clinician is a window (not shown) that provides the user with a history of a particular patient's medical history and a history of the various readings taken of that patient's vital signs. This history of the patient's vital signs (from previous readings taken by mobile users) can allow a clinician to quickly determine, at a glance, whether the current readings are within acceptable parameters or not; these displays enable the clinician to contextualize the data recently collected. By quickly comparing the current readings taken by the mobile user with the historical data, the clinician can determine whether further confirmatory readings are required or whether a dangerous condition is occurring. It should be noted that if the clinician determines that at least one reading is not within acceptable parameters, they may direct the mobile user to take more readings to determine if the previous readings were accurate.

Again regarding the dashboard, the current readings or data entries for each patient can be provided side-by-side with the historical data for that same patient. A side-by-side comparison allows the clinician to quickly determine if the new data is within acceptable parameters of the historical data. Moreover, any outstanding instructions to the mobile user can be shown on the dashboard adjacent to the current readings.

Referring now to FIG. 4B, another partial view of an navigational menu within an information window is provided. In this figure tabs are depicted that allow the clinician to navigate from one function of the directing clinician workstation to another. In this example, the tabs allow the clinician to quickly navigate between various information pages related to the patient LINDA FIRST. It will be appreciated that the tabs may be configured to allow the clinician to navigate to any page and/or function provided to the directing clinician workstation.

Referring now to FIG. 5A, a portion of an example directing clinician dashboard is depicted. The directing clinician dashboard is configured to be displayed on the directing clinician workstation 2. The directing clinician dashboard may be implemented as one or more web pages on a web server. Alternately, the clinician dashboard may be implemented as an application to be run on a general purpose computer such as a personal computer.

FIG. 5A depicts, at least in part, a patient management display and a part of a summary screen. In this example the patient management display displays the patients currently being directed and or observed under the “Directing” and “Observing” headings. Recently managed patients may also be displayed under the “Recent” heading. The patient management display is also configured to provide alerts to the clinician. Alerts may be raised for a variety of reasons that include, but are not limited to, when a directed user requires assistance, when a directing clinician has requested the administration or performance of a certain tasks by the directed user, emergency situations by the directed user, or when a timer for a reminder has expired. In the example depicted in FIG. 5A, the alert (as shown at the top of the patient management display) indicates that a directing nurse is required for the patient AGNES SMITH. The clinician may then click on the AGNES SMITH button to start directing care for this patient.

FIG. 5A further depicts a portion of an information window. In this figure side tabs are depicted that allow the clinician to see the statuses of all their open patient records and quickly context-switch between patients, keeping all tools for patient direction within the patient record tabs. These detail pages also include, but are not limited to, shift details, shift instruction, clinical note, shift tasks, review & end shift, and chat room. It would be understood that other tabs to other detail pages could be added without departing from the scope of this disclosure.

FIG. 5B shows various indicators that can be displayed next to a patient's name, according to one embodiment of the system. FIG. 5C illustrates a grey indicator with a number indicates the number of open instructions plus a count. These are the number of instructions that have been sent to a technician, which are still in an active status. A yellow indicator with a number indicates the number of forms that have been submitted by a technician and are waiting to be reviewed by the Directing Clinician. A red indicator with a number indicates urgent items and a question mark next to a patient's name indicates that a Care Team member has not yet arrived for that patient during the Clinician's shift (the Clinician whose user interface is being shown in FIGS. 5A and 5C).

FIGS. 5A and 5C also illustrate the dashboards wherein a clinician at a directing clinician workstation can also have observing permissions for one or more mobile users (eg., technicians) who are directed, supervised and managed by another clinician at separate directing clinician workstation. The observing status provides the observer with a display for that specific mobile user/patient, but does not permit them to direct the mobile user (e.g., technician), although the observing clinician does get access to the patient's chat room along with the directing clinician and the mobile user (e.g., technician). In this manner, should an issue arise whereby the observing clinician needs to take control the technician, they can do so seamlessly. This functionality assists with workload balancing between the various directing clinician workstations.

Referring now to FIG. 6, a partial example user interface following the workflow of communication between a Directing Nurse (DN), as displayed on the mobile of the technician 520 and the directing workstation of the DN is depicted. In this diagram, snippets of the user interface (UI) of the directing workstation of the DN or the mobile device of the technician 520 is shown. In this example, the status of the instruction may be displayed to the DN on the directing workstation. For instance, once the instruction has been sent 503, the instruction state on the UI may be shown as sent 602, accepted (506, 507), rejected 508, not done 512, and/or completed 511 depending on the state of the workflow. Alert notifications for the mobile device corresponding to whether an instruction has been sent 504 and/or whether an instruction has been cancelled 515, are also provided. An example UI of the mobile device 604 is shown demonstrating how a technician 520 might accept or reject an instruction. The UI may also display notes associated with the instruction. An UI excerpt is also shown regarding how a user enters data into the form associated with the instruction 509 and sends it to the DN via a network. In some instances, once the operation has been completed metadata associated with the workflow may be stored in the sever/workstation/data store as shift history information.

### The Relational Database and Relational Database Management System

In one embodiment, the Server/Database 6 comprises a relational database. The relational database stores data in the form of tables and uses a standard data query language (SQL) to execute data queries. Standard relational databases can store many different types of data in different types of tables, but retrieving data from diverse table sets can present challenges.

A relational database management system is used to create the structure in the database (create the tables and the relationships between them) as well as to input and retrieve the data. In one embodiment PostgreSQL is used as the database management system. Software code is written to insert data into the various tables in the database as well as to query the database to and generate reports comprising sets of data. In one embodiment, the Server/Database comprises an application server such as Tomcat and executes code written in a language such as JAVA.

### Entity, Attribute, Relationships, and Primary Keys

An entity represents a person, place, thing, event, piece of data, etc., that is to be tracked in a database. Each entity is recorded as a table in a database (e.g., patients, clinicians and patient medication record) along with the information relevant to each. Each table therefore represents a category of data to be stored and each row is comprised of a set of fields or attributes, which describes what information is being stored about that category of data. Entity and table can be used interchangeably in database design terminology.

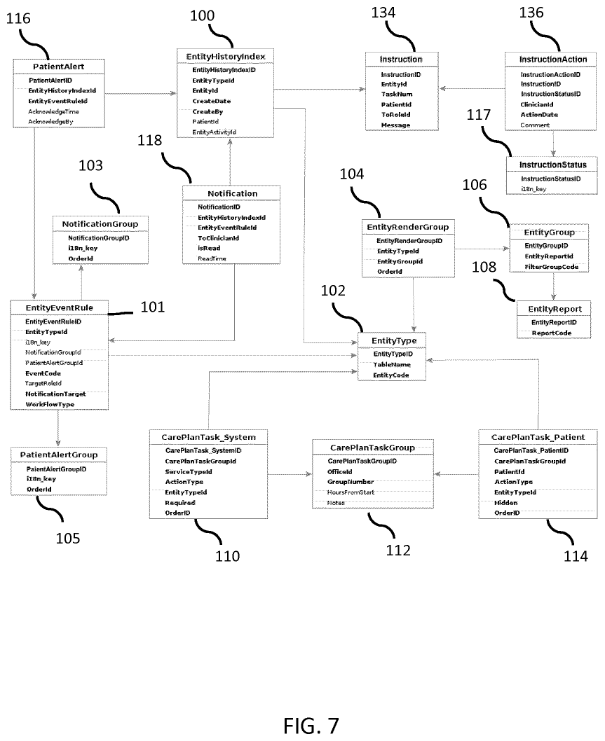

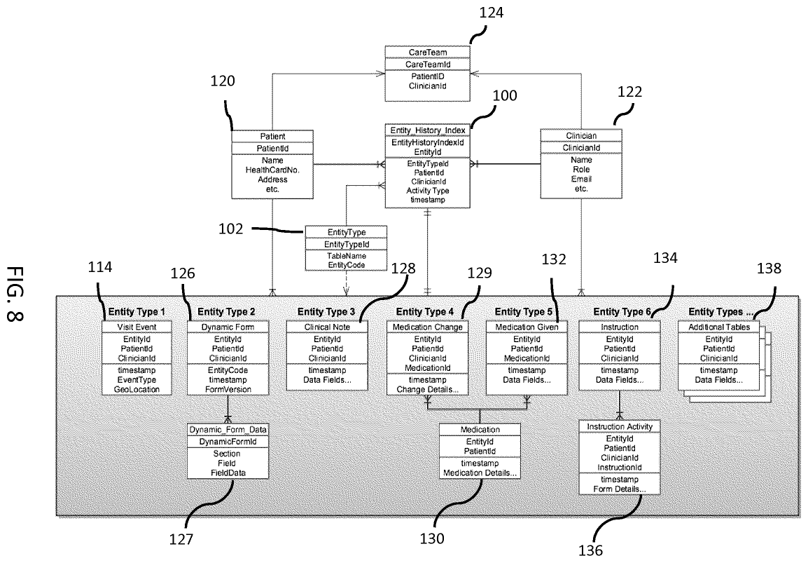

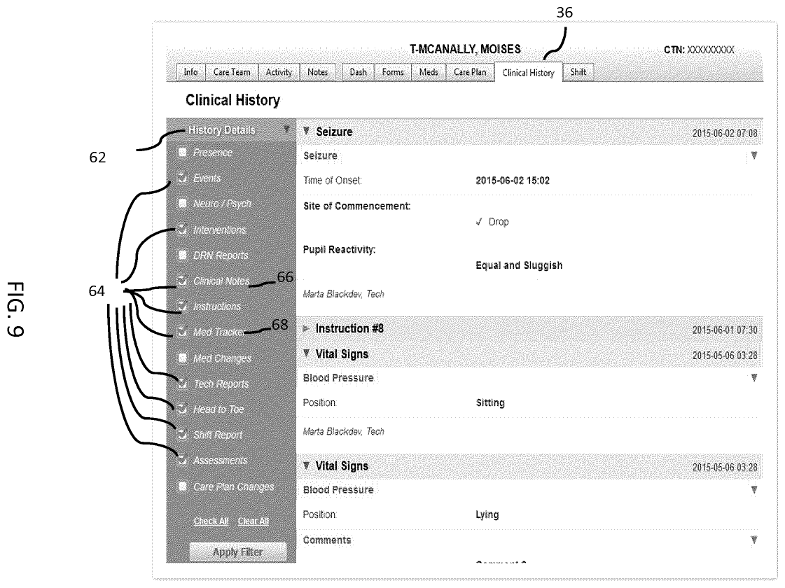

For example, with reference to FIG. 7, the table titled Patients 120, will contain the same type of information pertaining to all the patients (e.g., first name, last name, date-of-birth, phone number, etc.). Likewise, a table titled Clinician 122 will contain the same type of information pertaining to all the users of the system, which in one embodiment includes the technicians, therapists, therapist assistants, administrators, etc. (e.g., first name, last name, phone number, user ID, etc.). This table could be titled User and the individuals could be given a UserId, but for the purposes of this description the term Clinician is used for User. The table for patient medication could be called Medication 130 and could contain information such as the name of the medication, the prescribed dosage, the patient ID, etc.

Each occurrence of an entity is called an entity instance and becomes recorded as a record or row in the relevant table. Each patient, clinician, or medication is considered an entity instance within their respective table. An attribute describes various characteristics about an individual entity, which become the columns in the table (e.g., first name, last name, etc. are the attributes for each instance of an entity—patient or clinician) wherein each column represents a field of the record. The contents of each attribute can only take values from a given set; this set of permissible values for a column is called a domain. The titles of the columns are indicated in the rows of an entity relationship diagram such as illustrated in FIG. 7.

A primary key is an attribute or group of attributes used to identify an instance of an entity. In this system a primary key used to assign a unique identifier to each entity instance in a table, so each patient or user will be assigned their own unique primary key, which will be recorded in the entities' table (i.e., PatientId in Patient Table 120 or ClinicianId in Clinician Table 124) and can then be used by the system to find the information (attributes) for that individual by inserting it into another table such as the Entity History Index Table 100, where it is named a foreign key. Foreign keys do not need to be unique, for example the unique clinician primary key will be inserted into the Care Team Table 124 (becoming a foreign key) for a number of different patients.

Relationships define how one or more entities interact with one another. This is accomplished by creating a link between the relevant tables. In one embodiment of this system illustrated in FIG. 7, a link is created between the various clinicians assigned to a patient by creating a table titled Care Team 124 and inserting the primary key for each clinician along with the primary key for each patient (which would both be foreign keys in this table) along with a primary key for each entry). As illustrated in FIG. 8, a relationship is created between the Entity History Index Table 100 and the various tables 114, 126, 128, 129, 130, 132, 134, 136 and 138 because the EntityId and PatientId that appears in each of these tables also is recorded in the Entity History Index Table 100.

### The Entity History Index Table

One aspect of the system's relational database is the Entity History Index Table, which functions as a ledger that records patient data and events pertaining to the care of the patient. Examples of the types of data that are logged by the Entity History Index include patient: symptoms, vital signs, medications prescribed, medications given, consent; members of the care team, clinical notes, instructions given to a technician by a clinician, etc. Examples of the types of events that are logged by the Entity History Index include: when a patient's record was accessed and why, when an instruction was sent from a nurse to a technician, when the technician responded, when a user of the system conducted a consultation about a patient or a collaborative patient review meeting was held; etc.

The comprehensiveness of the data recorded in the Entity History Index and it's related tables supports the generation of a unique patient electronic record, which contains the detailed chronological history of the provision of the care to a patient in addition to unusually extensive amounts of detailed patient data. The configuration of the Entity History Index and it's affiliated tables also enables health care information to be provided, which excludes a patient's personal health information (PHI). The entire dataset that is directly and indirectly associated with a PatientId can be considered to the electronic “patient record” and segments of the data can be considered to be the various reports and views of the patient record.

In one embodiment, the system also comprises digital data such as scans, images, photographs, etc, although they are not specifically described herein. Digital documents are indexed and stored in an encrypted format external from the database linked to the electronic patient record by a shared unique patient identifier.

Referring now to FIG. 7, which illustrates some of the conceptual relationships between the Entity History Index Table 100 and some of the tables that the system uses to efficiently store patient data and to retrieve it in order to generate specific views of the patient record: the Entity Type Table 102; the Entity Render Group Table 104; the Entity Group Table 106; and the Entity Report Table 108. As will be described in detail below, this group of tables presented in FIG. 7 demonstrates that many different types of patient data can be collected in as many tables as practical for the system to manage (through the relationship between the Entity History Index 100 and the Entity Type Table 102). It also shows that the system keeps track of the communications between the various users of the system (within the Patient Alert 116 and Notification 118 subsystems in combinations with other tables within the database) and that this information is included within the patient records. FIG. 7 also shows that the system keeps track of when instructions are trafficked between the directing clinician and a technician (Tables 113, 115 and 117). Traditional patient records do not have the capability to capture, manage and display (render diverse datasets into a sequential historical record of care) this breadth and depth of information pertaining to the patient and the details of their care. In one embodiment, the patient record generated by this system is called an Electronic Community Care Record (ECCR), because it includes the details of the timing of the delivery of the care and communication involved in addition to extensive details about the patient's physiological (and mental/emotional) status.

One central aspect of the system comprises two tables, the Entity History Index Table 100 and the Entity Type Table 102, illustrated in FIG. 7. Each entry logged into the Entity History Index Table 100, includes: an EntityHistoryIndexID (the unique primary key for each entry into the Entity History Index Table 100); an EntityTypeId, and an EntityId (which indicates the table and row location of the data being referred to); a CreateDate; a CreatedBy; a PatientId and optionally an ActivityTypeId. This logical structure enables the system to quickly identify the relevant information for each entry in the Entity History Index Table 100 to quickly access and reassemble all the various types of records created in the delivery of care for a patient. A standard relational database structure, relying only on primary key/foreign key relationships could reassemble the historical record of care for a patient using PatientId and CreateDate (timestamp), but the amount of searching the system would need to conduct in order to reassemble the relevant records each time such a report is to be generated would be enormous and prohibitive for a system in actual use.

Thus, in addition to the patient's primary key (eg. PatientId), each entry in Entity History Index Table 100 includes a table location code (EntityTypeId) indicating the table or table sets where the relevant patient data resides and a unique key (EntityId) as a foreign key, which indicates the record in the relevant table (where it is a primary key) for that data, generally if the record pertains to a piece of patient data. This configuration allows for recording the pointers to all the patient data added into the database in a chronological order. Since each entry in the Entity History Index Table 100 includes an EntityTypeId (and an EntityID), the system refers to the Entity Type Table 102 to determine the table name for a table. In one embodiment, the system uses the name of the table, to find the table containing the data. When the EntityTypeId refers to a template form such as a form generated using the Dynamic Forms System, the Entity Code is used by the system to indicate which specific form was used to collect the data. For example, in FIG. 7, the Entity Type would indicate Type 2, a Dynamic Form Table 126, and the Entity Code would indicate the specific table such as “Vital Signs,” “Neurology,” “Pain Assessment,” etc.

### Entity History Index and EntityID: Patient Data and Information

One aspect of the invention provides a means to store patient data in many diverse table sets representing, for example, medications, assessments, instructions, clinical notes, etc., and the ability to be able to retrieve these records for a patient in chronological sequence in order to provide a record of the care given to the patient over a period of time. Means are also provided to be able to filter the information for a specific type of record and to be able to render the information according to different criteria.

In one embodiment, wherein the patient data is gathered using one of a plurality of forms (web pages) saved in a plurality of tables, the table location code in the Entity History Index Table 100 is given the label EntityId. Referring to FIG. 8, which illustrates one embodiment of the conceptual relationship between the Entity History Index 100 and a plurality of different types of tables containing patient information and data. The use of the EntityId enables each record in the Entity History Index Table 100 to have an additional relationship with one designated table in the system, where the data can be found within table type 1 or table type 2, or table type 3, or table type 4 and so on. The reference of EntityTypeId to the Entity Type Table, which provides the TableName and EntityCode enables the system to quickly identify in which table the relevant data being referenced is found and the EntityId key points to the specific record in that table.

In the conceptual example of how the Entity History Index Table 100 relates to the different types of tables through an EntityId presented in FIG. 8. In this example, the table titled View Event 118 is presented as Entity Type 1; the table titled Dynamic Forms 120 is presented as Entity Type 2 and points directly to the table Dynamic Forms Data 122; the table titled Clinical Notes 124 is presented as Entity Type 3; the table titled Medication Change 126 is presented as Entity Type 4; the table titled Medication Given 128 is presented as Entity Type 5 and both the Medication Change 126 and the Medication Given tables point directly to the table titled Medication 130; the table titled Instruction 130 is presented as Entity Type 6 and points directly to the table titled Instruction Activity 132; and FIG. 4 indicates that a plurality of other tables 136 also exist, wherein each type of table will have it's own unique Entity Type number, table name and code. The Patient Table 106 and Clinician Table 116 do not require an EntityTypeId, since no activity in these tables is tracked through the Entity History Index Table.

### Entering Patient Data

The process of entering & storing data can be described in conjunction with FIG. 8, which illustrates one embodiment of the conceptual relationship between the Entity History Index 100 and a plurality of different types of tables used to store patient information and data. As will be described in greater detail below, patient data is collected through the use of forms. A “form” is a web page displayed on the user interface of a computer, mobile phone, or other such device.

In this example, the table titled Clinical Note 128 in FIG. 8 is indicated to be an Entity Type 3. The form titled Clinical Note comprises a text field, wherein a clinician can enter an interpretation of the patient's condition into the text field after reviewing the patient information being provided by the technician caring for a patient. When the clinician enters their comments into the text field of the Clinical Note and saves the data, the system will log an entry into the Entity History Index Table 100, and an entry will be made into the Clinical Note Table 128 along with the PatientId, a timestamp, an EntityId (which functions as a primary key along with the PatientId to assist the system to find the entry in the table when queried) and the text of the clinician's commentary in the Clinical Note Table. The entry logged into the Entity History Index Table 100, will include an EntityTypeId (for example 00003) indicating that the data is stored in the table type 00003. The Entity Type Table 102 will have an entry for 00003, which specifies the TableName of “Clinical Note” and the entity code of “CLINICAL_NOTE.”

FIG. 8 also has a table titled Medications Given 130 and has been designated Entity Type 5, so for example the EntityTypeID would be 00005, indicating that the data is stored in the table type 00005. The Entity Type Table 102 will have an entry for 00005, along with an entry for the name “Medications Given” and an EntityCode of “MEDS_GIVEN.” The Medications Given Table 130 would have an entry comprising information such as, but not limited to, a MedicationId, indicating the record in the Medication table 130 describing the medication, the time the medication was given, any comments about the administration, the PatientId and the EntityId, which indicates which entry in the Medications Given Table 130 the system is searching for. EntityId functions as a primary key in the Medications Given Table 130 (along with the PatientId) and a foreign key in the Entity History Index Table 100 to assist the system to find the relevant entry in the Medications Given Table 130.

FIG. 8 also has a table titled Medication 130 and has been designated Entity Type 7, so for example the EntityTypeID would be 00007, indicating that the data is stored in the table type 00007. The Entity Type Table 102 will have an entry for 00007, along with the TableName value of “Medication” and an EntityCode of “MEDS”. The Medication table 130 would include the details about the medication such as, but not limited to, the name of the medication, the prescribed dose, the period of time it is to be given, the PatientID, an EntityID and any other information deemed to be relevant to the record. One skilled in the art would appreciate that additional information pertaining to the medication could be included in additional tables that could be related to the Medication table 130.

### Viewing Patient Data

There are a plurality of types of views of data that can be rendered on a workstation or a mobile device. The views are implemented in the application server based on use case and user role. Some of these views are indicated as tabs on the dashboard of the directing clinician workstation as shown in FIG. 4B, where the tabs are: Info 20 (Core Patient Info); Care Team 22; Activity 24 (Activity History); Notes 26 (Agency Notes); Dash 28 (Patient Dashboard); Forms 30; Meds 32 (Records of Medication); Care Plan 34; Clinical History 36; and Shift 38.

Non-limiting examples of the types of views (groups of tables queried and displayed) that can be generated are:
1) The Patient Activity History, accessed by the Activity Tab 24 in FIG. 5A. This report indicates the chronological historical record of care without exposing clinical data and would include data from any table containing patient data embodied by the tables in FIG. 8 such as Visit Event, Dynamic Form, Clinical Note, Medication Given, etc. This view would be used by accounts without permission to see clinical information such as a user administrator;
2) The User Activity History, reporting the chronological historical record of each time that a specific user accessed, updated or added to a patient record and for what reason.;
3) Clinical History—Web, accessed by the Clinical History tab 36 in FIG. 5A. This reports the chronological record of all patient care activities, data collected, and patient record changes displayed on a workstation. The Clinical History will display the data obtained in the template forms;
4) Clinical History—Mobile this report is similar to Clinical History Web, but presented on a mobile device;
5) Family Portal History shows the chronological record of aspects of the patient record that have been authorized by the patient for the family members to view;
6) Patient Forms Tab, accessed by the Forms Tab 30 in FIG. 5A, indicating the specific forms that have been authorized for use with each patient (See FIG. 24 for a COPD example);
7) Care Plan Tasks showing the specific tasks, which have been specified for a patient;
8) Instruction—Forms; and
9) Instructions—Delegated Acts.

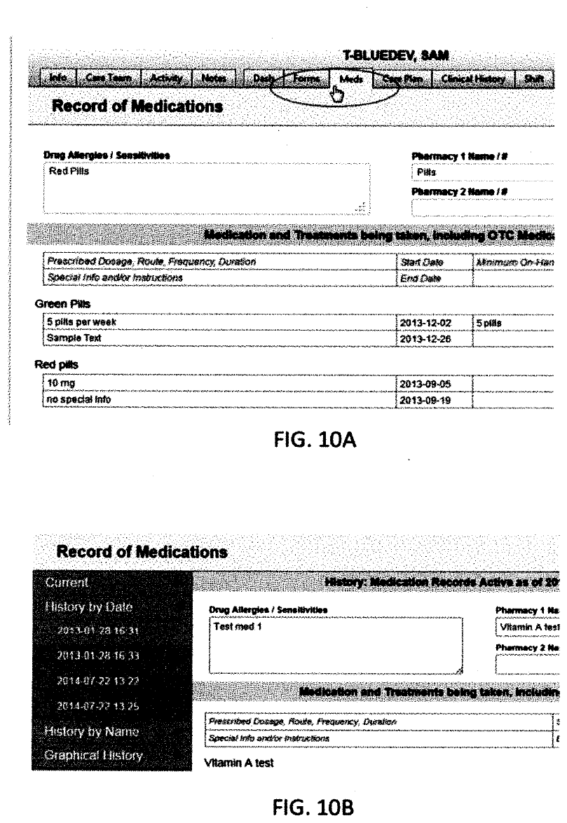

One method for how the system can retrieve data and present it in a view or report for a user of the system will be described with reference to FIG. 9, FIGS. 10A and 10B. When a user of the system wants to view some of the information in a patient file, the system will render the data on the user interface of the user's device. In one embodiment, this presentation of information is called a report. If, for example, a user of the system wants to review all of the clinical notes and medications that have been submitted for a patient over the past week, they would submit a request to the system to render a report by selecting an appropriate indicator (icon) on the user interface of their device for that report.

To view the medications given and the clinical notes, a user would know that these types of patient data are presented within a Clinical History. FIG. 9 depicts an example of a user interface on a workstation of a directing nurse, wherein the nurse has clicked on the tab for Clinical History 36. This Clinical History user interface corresponds to the view titled Clinical History—Web. The History Details 62 presents a number of options to the nurse: Presence; Events; Neuro/Psych; Interventions, DRN Reports; Clinical Notes 66; Instructions; Med-Tracker 68; Med Changes; Tech Reports; Head to Toe; Shift Report; and Assessments; Care Plan Changes. In this example, the nurse has selected certain of these options as indicated by check 64 in the box adjacent the title. Each of these options presented under History Details 62 will present data to the nurse that has been stored in the appropriate table or cluster of tables in the relational database.

FIG. 10A depicts an example of a user interface on a workstation of a directing nurse, wherein the nurse has clicked on the tab for Meds 32. FIG. 10B illustrates the example presented in FIG. 10A, wherein the nurse has filtered the results by date.

According to one embodiment illustrated in FIG. 7, there are a group of tables pertaining to the ability to render different kinds of views of patient data: the Entity Render Group Table 104; the Entity Group Table 106; and the Entity Report Table 108. The Entity Render Group Table 104 groups the various tables to be queried for each type of report, wherein each table is identified by its EntityTypeId. Some tables can be queried for multiple reports so they would be listed multiple times. The OrderId designates the order in which table in a group is to be queried. Each grouping of tables is identified by an EntityGroupId. The Entity Group Table 106 associates each EntityGroupId with an EntityReportId and a FilterGroupCode, which is used to denote the language the report is to be displayed in. The Entity Report table 108, is used to associate each EntityReportId with a ReportCode, which is used by the system to assign a name to each EntityReportId.

In a hypothetical example pertaining to Clinical History—Web, there are 11 possible types of reports, represented by 11 different groupings of table data (each available in English, French, Russian or Spanish), which equates to 44 unique reports (11 reports in 4 languages). The Entity Report Table 108, would have 44 different ReportCodes one of which would be “Clinical History—Web” associated with an EntityReportId of 00041.

| EntityReportId | ReportCode |
|---|---|
| 00041 | Clinical History - Web |

The Entity Group Table 106 associates each EntityReportId with an EntityGroupId and a FilterGroupCode (in this example English), indicating the language the report is to be rendered in (this table would have 44 EntityReportIds, 11 EntityGroupIds and 4 FilterGroupCodes: English, French, Russian or Spanish. A segment of the Entity Group Table 106 table is represented by:

| EntityReportId | EntityGroupId | FilterGroupCodes |
|---|---|---|
| 000037 | 000010 | English |
| 000038 | 000010 | French |
| 000039 | 000010 | Russian |
| 000040 | 000010 | Spanish |
| 000041 | 000011 | English |
| 000042 | 000011 | French |
| 000043 | 000011 | Russian |
| 000044 | 000011 | Spanish |

The Entity Render Group Table 104 would list the various combinations of the tables using the EntityGroupId and the OrderId indicates the sequencing of the tables in the report. A segment of the Entity Render Group Table 104 table is represented by:

| EntityRenderGroupID | EntityTypeID | EntityGroupID | OrderID |
|---|---|---|---|
| 0000001 | “Neuro/Psy” | 000011 | 00003 |
| 0000002 | “Presence” | 000011 | 00001 |
| 0000003 | 0000??? | 000010 | 00002 |
| 0000004 | “Events” | 000011 | 00002 |
| 0000005 | 0000??? | 000004 | 00001 |
| 0000006 | 0000??? | 000006 | 00001 |
| 0000007 | 0000??? | 000010 | 00001 |

In this example, the system would know that the Clinical History—Web report pertains to EntityGroupId 000011 and it would list the entries specified in the group. The group is composed of a plurality of EntityRenderGroup entries which specify the various forms which are members of the group. The EntityRenderGroup designates the form by EntityTypeId corresponding to the EntityTypeId indicating Events first, then the table corresponding to EntityTypeId indicating Interventions and then the table corresponding to EntitytypeId indicating Clinical Notes and so on.

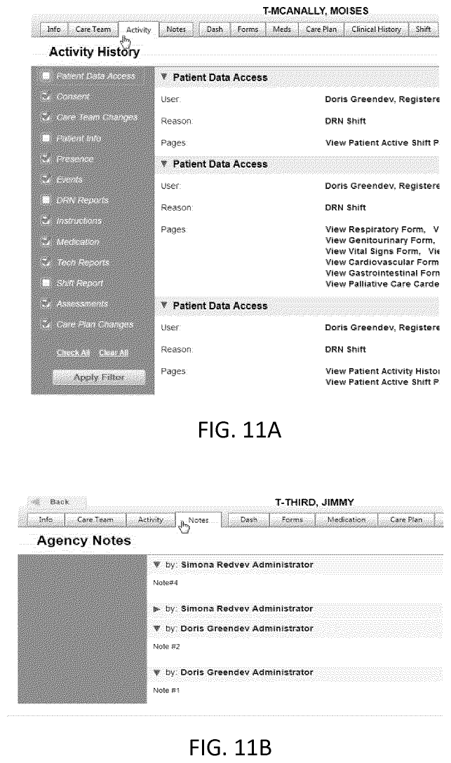

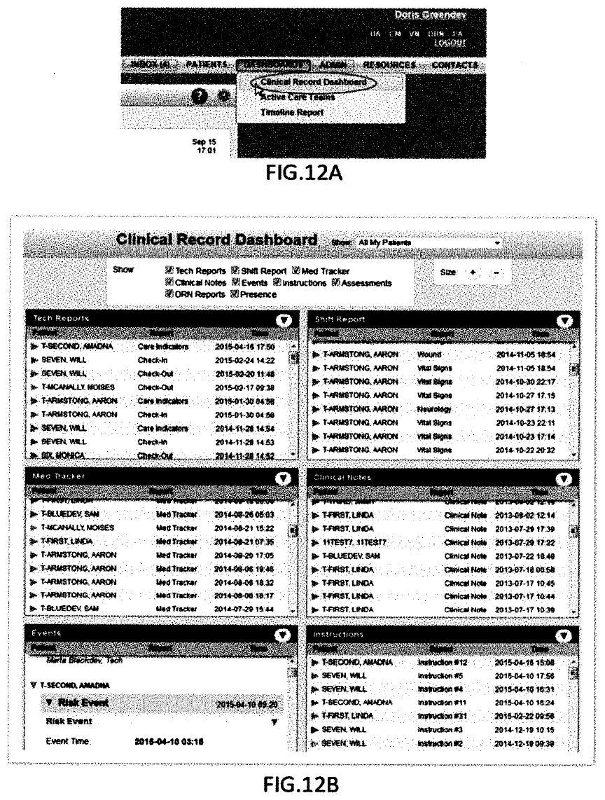

Examples of other views—FIGS. 11A and 11B—Notes tab. FIGS. 12A and 12B show views accessed from the main menu pertaining to Dashboards, wherein FIG. 12B illustrates one example of a Clinical Record Dashboard.

The system can also retrieve entries made by a certain clinician, technician or other authorized user of the system by searching the Entity History Index Table 100 for all records containing a relevant PatientID, the relevant UserId in the CreatedBy field and the date range by searching the CreateDate field.

### Reports Excluding Patient Data

Views of the patient care activity can alternately be rendered by the application server for a patient without exposing clinical data and only showing the type of data that was entered such as the form title or activity type, the time of the activity, and who the activity was by. This non-clinical view can also be rendered by the application server for a specified user. These views would be most useful to users who have administrational roles which do not have access to clinical data.

### Notifications and Patient Alerts

The system is configured to issue patient alerts and notifications in response to specific types of events.

A notification is an electronic message that is sent to the inbox of a clinician with information that is relevant to them. Clinicians will receive notifications in their inbox, even if they are not actively on a shift. Notifications can include information such as if a change has been made to a Care Team, a requirement for a Directing Clinician on a shift, a new professional note on a patient, an LSA event on a shift, etc.

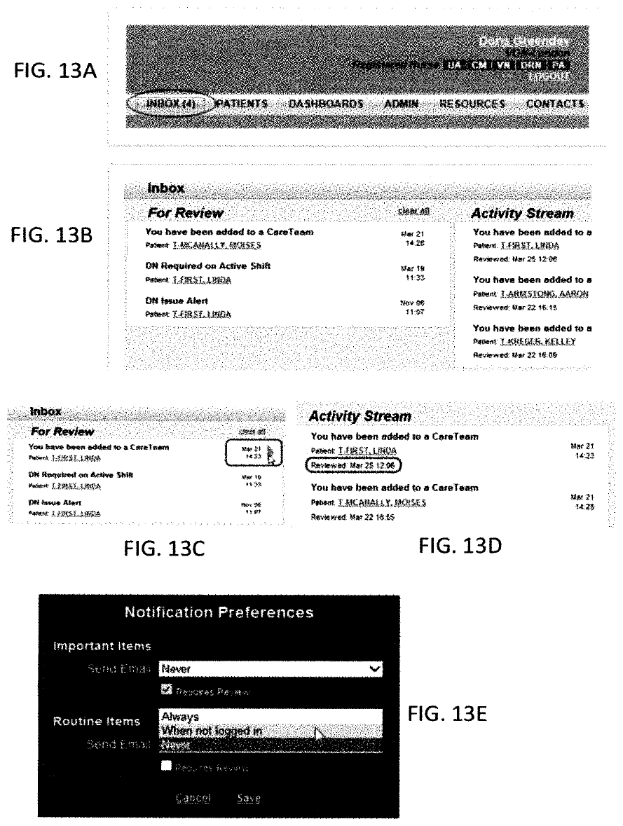

FIGS. 13A-E illustrate how notifications are presented on the user interface of a Directing Workstation.

A patient alert is a notification generated by about new information on a patient, which may be sent to the Directing and Observing clinicians or the Technician providing care to the patient. This information will also be reflected in a patient's Electronic Community Care Record so that, if requested, the system will be able to show who had the relevant information sent to them, how, when and why.

Referring to FIG. 7, information pertaining to patient alerts is recorded in the Patient Alert Table 116 and the Patient Alert Group Table 105. Information pertaining to notifications is stored in Notifications Table 118 and Notification Group Table 103. Both sets of tables record the relevant EntityHistoryIndexID. Which kinds of events qualify for patient alerts and notifications are defined by an Entity Event Rule Table 101 by inclusion of the EntityEventRuleID in a field of the Patient Alert Table 116 and Notifications Table 118. The Entity Event Rule Table 101 also relates directly to the Notification Group Table 103 and the Patient Alert Group 105.

### The Status of Instructions

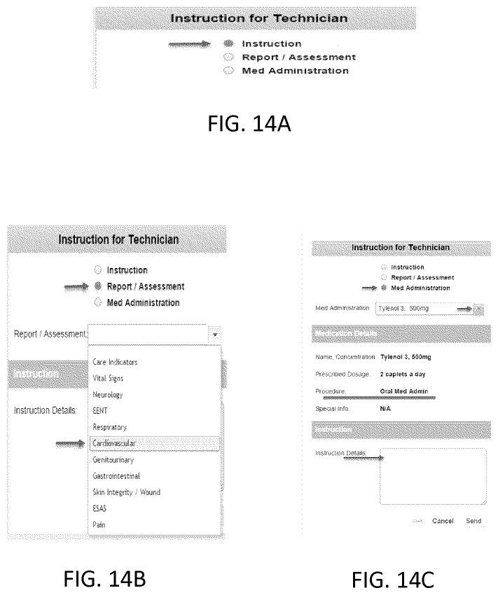

One specific type of a form is an instruction. Instructions include a piece of data indicating its status (e.g., sent, received, rejected, etc.). This aspect of the system was introduced with reference to FIG. 6. Referring to FIGS. 14A-C, which illustrate three examples of types of instructions which can be sent by a Directing Clinician to a technician: Instruction, Report/Assessment, and Med Administration.

There is a set of tables in the database recording information pertaining to instructions and the status of instructions as they are trafficked back and forth between a clinician (or therapist) and a technician (or therapist assistant), as illustrated in FIG. 6. These tables are shown in FIG. 7, titled Instruction Table 134, Instruction Action Table 136 and Instruction Status Table 117. For the purposes of this description, this group of tables is referred to as Instruction Group of Tables.

The Instruction Status Table 117 holds a list of all the status options for the system to assign to the status of an instruction in association with a unique InstructionStatusID. Examples of possible status options are: sent, received, rejected, cancelled, accepted, cannot complete, etc. Each time an instruction is sent, received and/or responded to an entry is made into the Entity History Index Table 100 relating to the Instruction Group of Tables, indicating the status of an instruction has changed. The Instruction Action Table 115 will indicate the status of the instruction by the InstructionStatusID, along with the date and time of the change in status (in the ActionDate field), the ClinicianID, optionally comments and the InstructionID. The InstructionID will refer to the original instruction, which is stored in the Instruction Table 113 (entityID, tasknum, patientId, ToRoleID, and message).

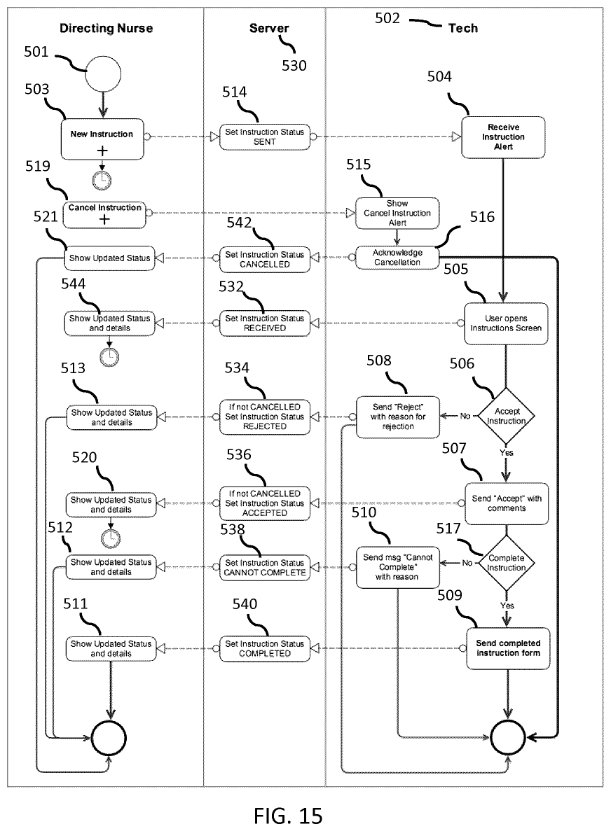

Referring now to FIG. 15, a process diagram for an example communications process between a directing nurse (DN) 501 and a technician 502 is provided. This process diagram describes the flow and state of instructions as they are passed from a directing workstation to a mobile device (or device being used by a technician). In this example embodiment, a server may be configured to mediate and/or traffic instructions between the directing workstation and the mobile device. The server 530 may include a data store and/or a relational database management system for storing and retrieving, at least in part, data associated with the system.

In this example, the DN 501 first issues a new instruction 503 on the directing workstation and it is stored in the Instruction Table 113 in the Server 530. The instruction 503 is sent, over the network, to the mobile device of the technician 502. The message may be mediated, routed, or trafficked by a server 530. The server is configured to store, at least in part, a status of an instruction 503. The status of the instruction 503 is indicated as “sent” in the InstructionAction Table 514.

Once the instruction 503 has been delivered, the technician 502 receives an alert 504 on the mobile device. The Java application queries the Instruction Tables to determine when instruction-related alerts should be sent.

The technician 502 then opens the instruction on the mobile device 505 to respond to the instruction 506. In this example once the instruction 503 has been opened on the mobile device, the server 530 updates the state information of the instruction to “received” 532 and stores it in the InstructionAction Table 514 of the server 530.

The technician 502 then has the option of either accepting the instruction 507 or rejecting the instruction 508.

If the technician rejects the instruction 508, then a message will be sent, via the network, to the server 530. Once the rejection 508 has been received at the server 530, the server updates the status information of the instruction to “rejected” 534 and stores the status, along with text details for the reason for rejection 513.

In this example once the server 530 has stored the information related to the state change in the data store, the server 530 is configured to send the updated status to the directing workstation. A status associated with the state of the instruction will be marked “Rejected” along with text details for the reason for rejection, 513 on a display of the directing workstation.

If the technician 502 accepts the instruction 507, then the technician 502 will be expected to later complete, at least in part, a form corresponding to the instruction. An “acceptance” message will be sent from the mobile device of the technician 502, via the network, to the server 530. Once the “acceptance” 507 has been received at the server 530, the server updates the state information of the instruction to “accepted” 536 and stores the state in the InstructionActionTable 514. The server 530 is then configured to send a message to the workstation of the DN, and a status associated with the state of the instruction will be marked “accepted” on the display of the directing workstation 520.

If the technician 520 completes the instruction, they fill-in the completed instruction form and hit “send” 509, then the entered data will be sent, via the network, to the server 530. The server 530 then stores data and metadata associated with the completed instruction form in the InstructionAction Table 514. The server updates the state information of the instruction to “completed” 540.

In this example the server 530 is then configured to send a message, via the network, to the directing nurse's workstation. A status associated with the state of the instruction will be marked “completed” on the display of the directing workstation 511.

If the technician 520 cannot complete the instruction, then the technician 520 completes the “cannot complete” form 510. A message will be sent, via the network, to the server 530. The server 530 then stores data and metadata associated with the cannot complete instruction form 510 (including a reason for the cannot complete state) in the InstructionAction Table 514. The server updates the state information of the instruction to “cannot complete” 538.

[12] In this example the server 530 is then configured to send a message, via the network, to the directing nurse's workstation. A status associated with the state of the instruction will be marked “Cannot Complete” 512.

In some cases the DN may wish to cancel the instruction 519. If the DN cancels the instruction 519 a message is sent, via the network, to the server 530. The server updates the state information of the instruction to “cancelled” 542 in the InstructionAction Table 514.

In this example the server 530 is then configured to send a message, via the network, to the mobile device of the technician 502. Once the cancel instruction message is received on the mobile device of the technician 502 a “receive cancel alert” 515 is displayed on the mobile device of the technician. Once the technician 520 opens the instruction screen 516, a message is sent, via the network, to the workstation of the DN indicating that the instruction has been cancelled 521.

It will be appreciated that the state information may be stored in the datastore or in a relational database of the server 530. In this example, the state information may be associated with a task or instruction information stored in the database. Furthermore it will be appreciated that the state information of the instruction may be used to determine the next permitted step or action. For instance, if the state of the instruction is “cancelled”, then any subsequent state changes (other than, in some embodiments, a “reactivate” state change) may be ignored. Impermissible state changes (such as from “Rejected” to “Accepted”) may also be ignored, or may trigger exceptions, alerts, warnings, or some other indication that an impermissible or illegal state change has been detected. This information may then be used by administrators to debug or troubleshoot the system.

### The Care Team and Parties External to the Care Team

In one embodiment, the Care Team comprises individuals organized by roles, who have clinical access to a patient record. Each role generally has its own unique set of permissions and functionalities that are relevant and appropriate to their responsibilities of caring for a patient. Different patients can have different roles involved in their care team. Different patients who have the same roles involved in their care can have different individuals in these roles.

Over the course of care for a patient, changes may occur to the Care Team and/or other individuals responsible for the care being provided to a patient. The roles of who are assigned to a patient could change, for example, as a patient progresses from early to late stage of a disease and requires different kinds of care during the progression. The individuals in the roles assigned to the Care Team for a patient can also change as shifts turn over and who will be available on the day and during the time allotted for the meeting. People may transfer to other locations in the system; people go on vacation, etc. The system keeps track of and makes changes to the individuals on the Care Team assigned to a patient to facilitate inviting the correct individuals to a meeting.

In one embodiment, the Care Team comprises a number of people who are assigned to a patient, which can vary from patient to patient.

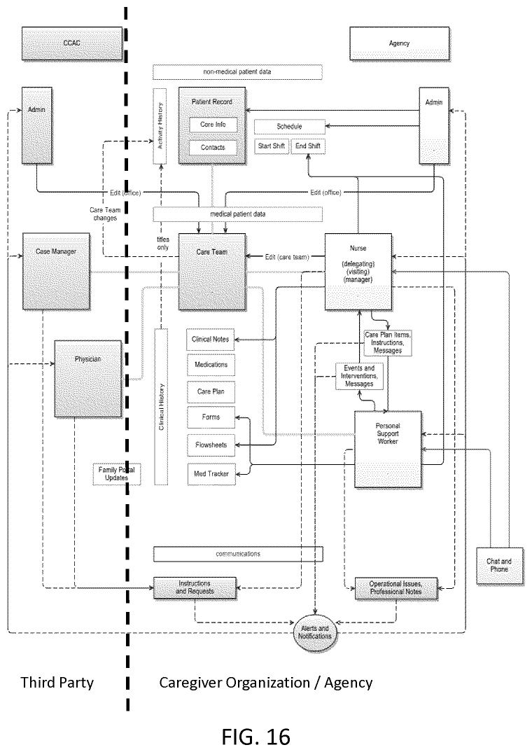

FIG. 16 illustrates one embodiment of a system configuration wherein the Care Team comprises members from a third-party organization, who are essentially observers watching the performance of the Caregiver Organization/Agency, which comprises the directing, observing and consulting clinicians, mobile users, administrators, etc., who are using the system.

Managing the communication includes, but is not limited to, storing and retrieving data entered into the system by any party; initiating, facilitating, and logging voice, chat, email, video, and/or blog communications between parties using the system; generating alerts and/or notifications and directing the alerts and/or notifications to the appropriate party; and/or generating, retrieving, and storing metadata of the data entered into the system and any communications initiated or facilitated by the system.

In this example, the system retrieves and stores data related to, among other things, technician schedules and their associated start and end shift times, nurse schedules and their associated start and end shift times, administrator schedules and their associated start and end shift times, the patient record, the patient's medical data, the patient's non-medical data, clinical notes, medications, care plans, forms, flow sheets, and medical trackers. The system is also configured to allow nurses, agency administrators, and/or personal support workers to access and/or modify some of the data stored in the system's data store.

In this example metadata based on the stored data is also captured by the system and stored in the system's data store. This metadata can be used to analyze and report on, among other things: the attendance of a nurse, admin, or personal care worker; a patient's clinical history; an activity history (and associated metadata such as date of administration, time taken to perform an activity, time to report an activity, etc.) of each activity performed.

The system may also be configured store contact information of each party and/or person that has access to the system's data store. This data would also be stored in the system's data store.

In the embodiment described in FIG. 16, other third parties, such as additional service providers (e.g., physiotherapy or psychological service providers, disease specialty consultants, extra insurers and alternative funders, etc.) may also communicate with the system and the parties in the Care Team. Third parties might include, but are not limited to, an insurer, government body, oversight committee, hospital network, public health researchers, or any group that may be interested in patient data, including anonymized patient data.

The system may also manage the data flow between the parties in the care team and the third parties. In some examples the third parties may only be permitted access to a subset of the data in the care team's data store (e.g., anonymized). This may be useful, for example, in protecting the privacy of a patient.

In the embodiment provided in FIG. 16, the third party may be allowed to observe, provide instructions, and/or make requests to the clinician (e.g., nurse) or the technician through the system. For example, a third party physician that is outside of the care team/agency may modify a drug dosage for a patient, which would then be communicated to the clinician, technician, or both, through the system. Similarly, a case manager may approve a treatment plan proposed by a physician and stored on the core team's data store. This approved plan might then be communicated to the clinician, technician, or both through the system.

It will be appreciated that, in some example embodiments, the system may contain several layers of licensed medical professionals, and that each layer may be capable of initiating communication with any other layer. For instance, in an example embodiment the system may also have a licensed physician, administrator, etc. who would occupy a higher layer in the hierarchy. The licensed physician (or higher layered individual) may be tasked with observing, managing, and/or directing any number of regulated or non-regulated personnel including, but not limited to, registered nurses, nursing assistants, and/or technicians. In some example embodiments the physician (or higher layered individual) may be logged into a directing clinician workstation. In other example embodiments the physician (or higher layered individual) may be logged into an observing workstation. In yet another embodiment, the physician (or higher layered individual) may be logged into an administration workstation.

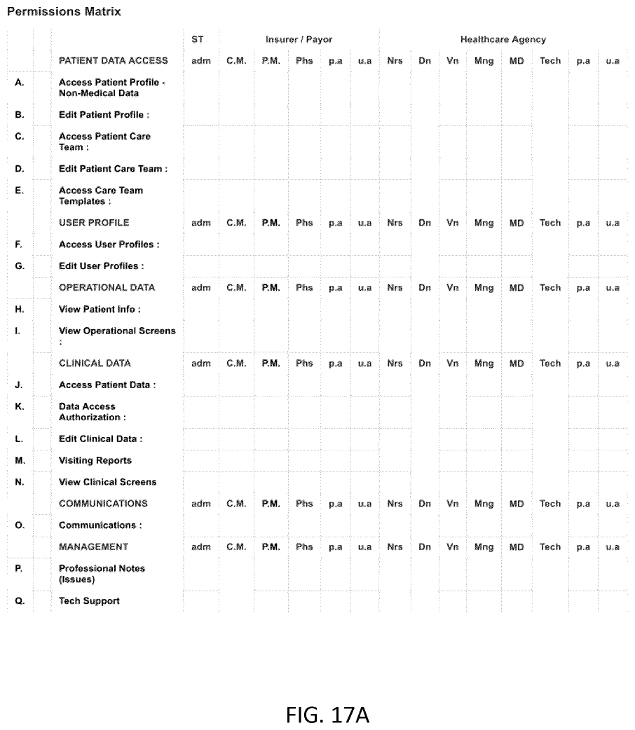

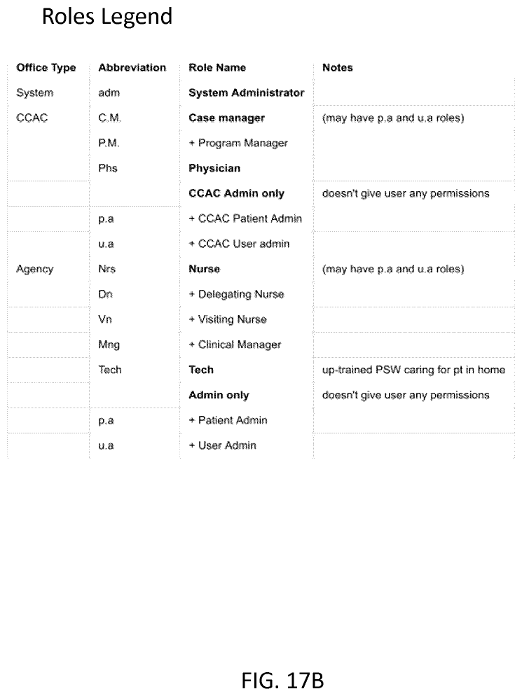

FIG. 17A illustrates some exemplary roles and possible permissions/functionalities that could be associated with the roles. FIG. 17B describes the legend of role names presented in FIG. 17A, which also illustrates some of the possible general roles that may be included in a Care Team for a patient. Note that CCAC (Community Care Access Center) is included as an example of an Insurer/Payor.

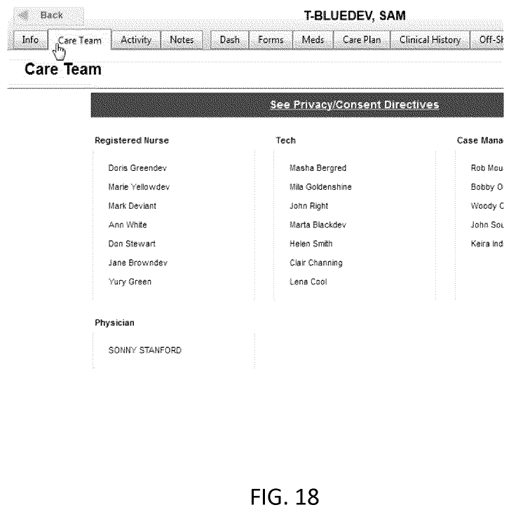

FIG. 18 illustrates one example of how the system keeps track of all the current members of a Care Team and presents the information to the users of the system such that they can easily identify who is on the Team and select them for information or to arrange a communication.

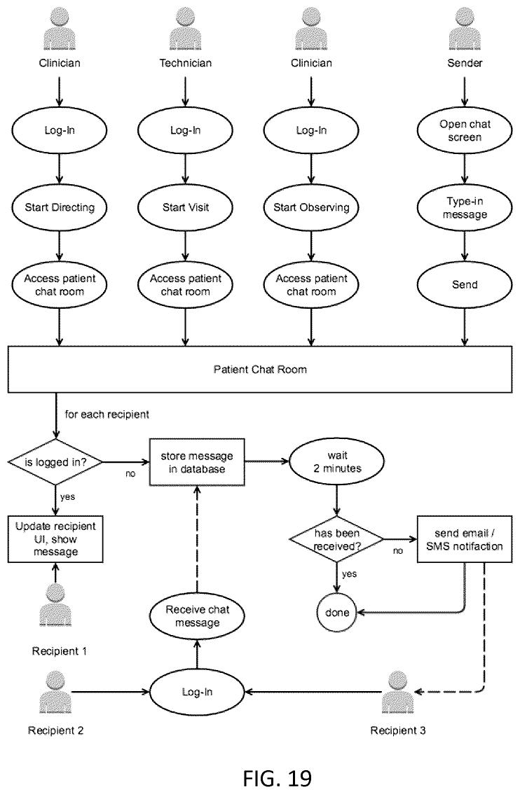

Users of the system can use the system via a private “chat room” associated with a patient's record. FIG. 19 depicts an example work flow diagram of a chat room functionality according to one embodiment of the system.

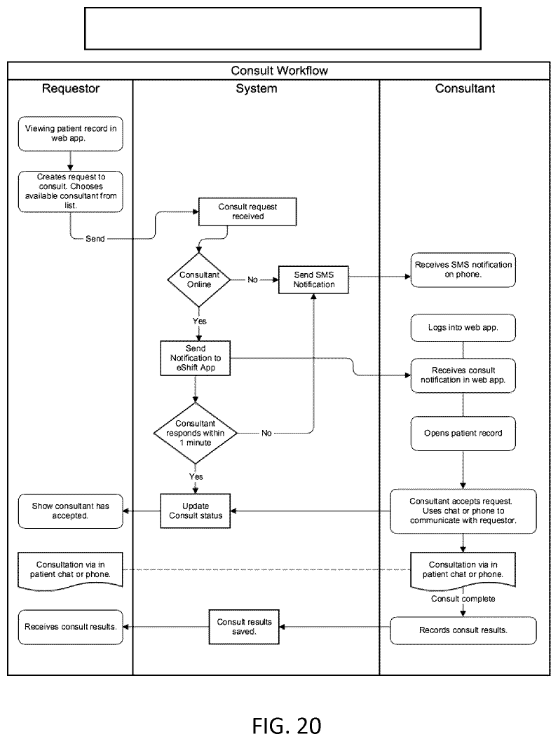

FIG. 20 shows an example workflow for a consultation, including the Requestor, the System and the Consultant according to one embodiment of the system.

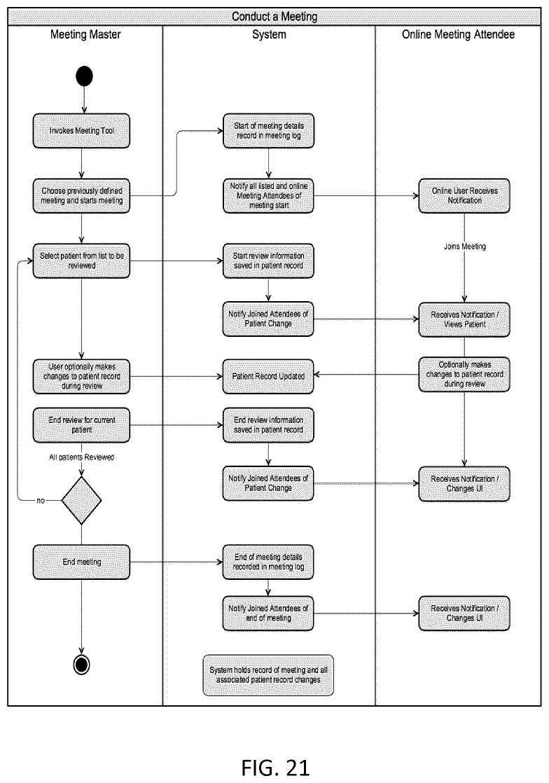

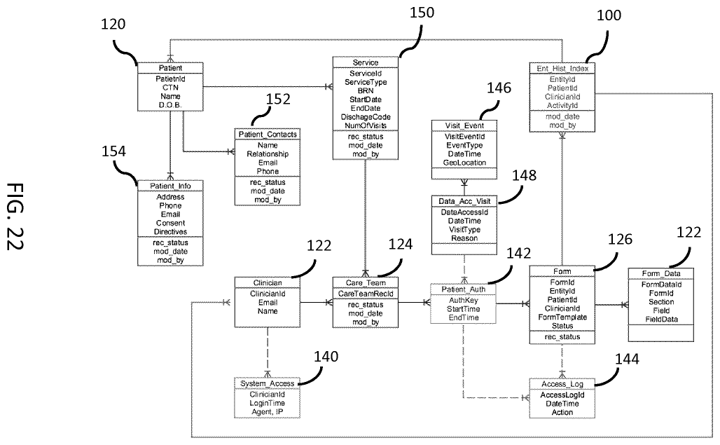

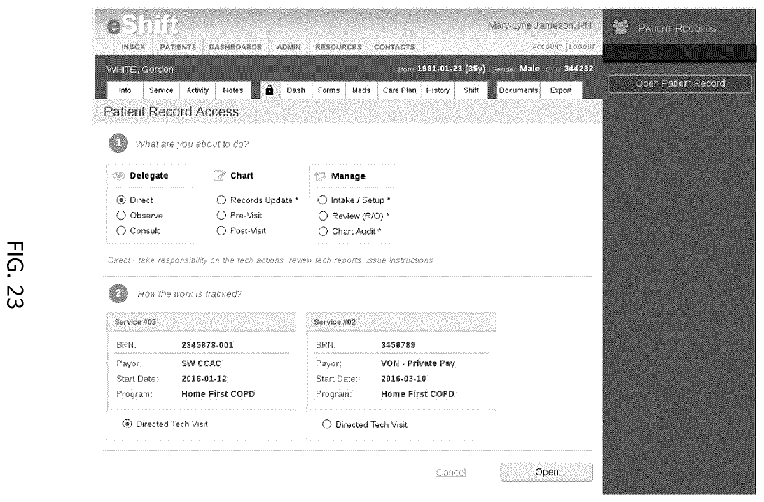

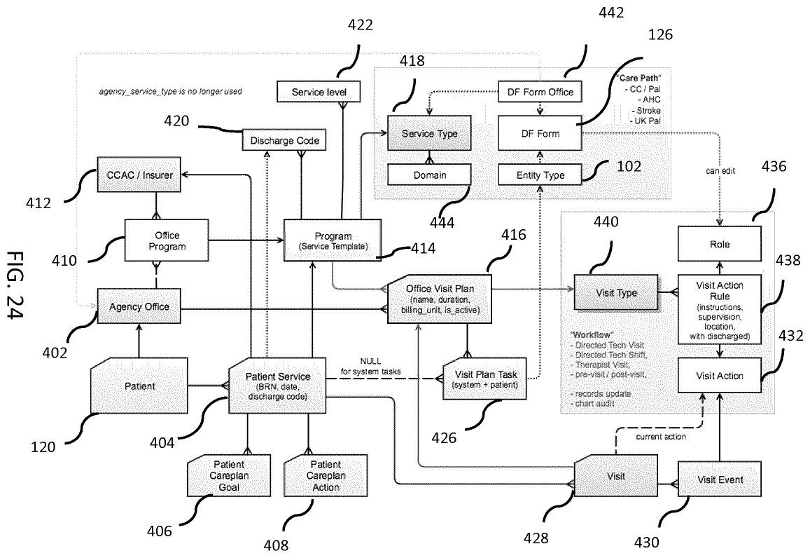

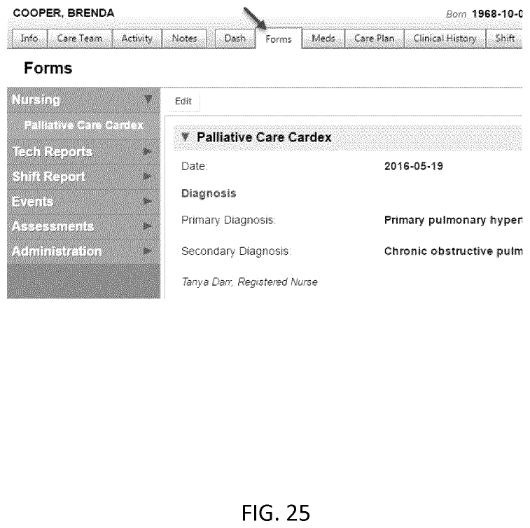

FIG. 21 describes, according to one embodiment of the system, an example work flow diagram for conducting a multi-party meeting, including the Meeting Master, the System and the Online Meeting Attendee.

### Authorizing and Recording User Access

In one embodiment, the system is configured to check access rights of the user, grant rights to access a requested patient, record the purpose for access, record each time a user accesses the patient record, for how long, and track what was viewed or altered. For example, if a nurse opens a patient record to view a Clinical Note, a record in table Data_Acc_Visit 148 will be saved indicating the purpose of access which was granted for a specific patient, an Entity History Index Table 100 record pointing to this record in Data_Acc_Visit Table 148 so that this users access appears in the clinical record of this patient, a record in Patient_Auth 142 which specifies the key which the browser will use to access the specific patient's data, and a record in Access_Log 144 which specifies which data was viewed, in this case, Clinical Note.

It will be appreciated that reports can be generated that associates an Entity History Index Table 100 entry with any other part of the system with which it has a direct or indirect relationship. For instance, a report could be generated that allows administrators to determine the Entity History Index Table 100 data that is specifically tied to an Access Log Table 144. In this example, reports including Entity History Index Table 100 data can be generated for any of the entities that the Entity History Index Table 100 is connected to, whether directly or indirectly.

### PatientServiceIds & Billing Reference Numbers (BRNs)

In one aspect, this system comprises PatientServiceIds, which attach to each session wherein an individual in an authorized role enters into a patient record to perform some service pertaining to the provision of health care to a patient. A session could include for example: a nurse entering a patient record in order to direct a technician caring for a patient; a technician entering a patient record in order to receive forms and instructions to provide care for a patient; an administrator entering a patient record in order to conduct a chart audit; a user seeking a consultation with another user; a number of users conducting a multidisciplinary meeting to discuss the patient's progress and Care Plan; a therapy session conducted by an assistant under the direction of a therapist, a care session conducted by a visiting nurse, etc.

PatientServiceIds interconnect who is providing a service, to which patient, and who is paying for the service, as each service can be conceptualized as arising from an approved “purchase order.” In some embodiments, when a PatientServiceId is used as a purchase order, it can be used to approve a block of clinical interventions, such as a set number of therapeutic visits. For example, in some embodiments, a PatientServiceId denotes eight approved visits by a speech language pathologist (or some other set number of therapy sessions).

PatientServiceIds are used internally by the system to track and approve who is entering a patient's ECCR, (who, when, why, how long) in addition to identifying the various roles associated with an approved Patient Service Plan. The internal PatientServiceIds are provided by and used by the system. The External PatientServiceId will generally be provided by the payor of the service, although in some cases can be provided by an agency providing the service, for example in situations when the patient will be paying for the service (e.g., extra therapy sessions). For the purposes of describing the system, the term, Billing Reference Number (BRN) will be used to denote the external PatientServiceId, although it is understood that a different name could be used. Other terms that could be used are Service Offer, Service Request, Service Order Number, Purchase Order, etc.

The BRN name and scope of services will depend somewhat upon how the delivery of health care services are funded in the jurisdiction in which the system is being deployed. The proportion of government versus private funding varies from country to country. For example, in Canada 70% of healthcare funding comes from the public-sector and the remaining 30% from the private-sector. Other western developed nations such as the Czech Republic, Denmark and Norway, public expenditures account for approximately 85% of total health care spending. At the other end of the spectrum, in countries such as the United States and Mexico, public spending accounts for only 45%.

Thus, in jurisdictions such as the United States and Mexico, health care funding and especially community based health care funding will generally be provided by more than one payor and each will have their version of External PatientServiceIds that they provide to track services, which they have pre-approved. In general, the payor(s) and the agency providing the bulk of the health care services (agencies may contract out to other health care service providers to provide specialty services, such as therapy services, etc.), will develop care plans designed for a class of patients (e.g., stroke versus COPD versus palliative) for which the payor agrees to fund.

Some ways that the US billing systems currently function are with “Super Bills,” which detail the discrete events/actions services with billing codes. The current health care system in the United States uses complex billing terminology based on Current Procedural Terminology (CPT), a system of numeric codes that has been developed and maintained by the American Medical Association (AMA) in connection with the Health Care Financing Administration (HCFA) Common Procedure Coding System. Using Current Procedural Terminology (CPT), medical services are described using numeric codes. These numeric codes have been established in the United States as the standard code set for reporting health care services in electronic transactions.

In order to bill a client in the United States, a physician has traditionally completed a superbill/patient encounter form after a patient's visit. This superbill has the diagnosis. This (ICD) code and the procedure, (CPT code) which describe the surgery or E&M code details of the encounter is required for billing the patient or insurance company. The office staff then fills out the insurance claim form (the HCFA 1500 form) manually for billing the insurance company, or the information and codes are entered manually by the office staff into a computer software system, which then creates a patient file. The office staff then can enter the appropriate billing codes into the insurance claim form (HCFA 1500), which is part of the computer system. This can then either be printed out and mailed to the insurance company or sent electronically to the insurance company.

The current system of Current Procedural Terminology (CPT) codes has become highly complicated. Appropriate definitions for the codes and accurate reimbursement amounts for each code have become increasingly difficult, and frequently change. In addition, a medical practitioner consumes an inordinate amount of time keeping up with the codes and associated record keeping, which leaves less time available for patient care. One embodiment, would be configured in order to “package” the allowable services under a BRN.

In one embodiment, the point of contact between the patient and the service provider will be the payor, who will submit a purchase order to the agencies they have contracted to provide health care services. In general, jurisdictions where the provision of health care is largely publically funded (e.g., Canada, Denmark, or Norway), the local government agency will provide codes for the services they have approved for funding. In Canada, the province is the insurer/payor and they contract with home care providers for the care of a patient. The order for care is a service order.

In one embodiment, the agency will submit a billing number/code to the payor(s) to request payment for services approved under the care plan for a patient. In one embodiment deployed in jurisdictions where the provision of health care is largely privately funded by a number of sources (e.g., as in the US) the agency will submit billing codes to the various payors.

In the United Kingdom, the period of time that is approved for patient care services is termed a “spell” or an “episode of care.” The administrators track each spell, which has an admission date and a discharge date. If the patient comes back after discharge, it is considered a new spell.

For example, in Ontario, Canada, the BRN functions as a purchase order for services. The Ontario government, the CCAC, informs their home care providers that a patient needs a certain type of care (no patient-specific data is sent with this offer). The home care provider will then accept or decline the offer. IF they accept it, they then receive a “Service Order” with the full details of the patient and the service with includes the “BRN” which is equivalent to a purchase order number. Community Care Access Centres (CCACs) arrange all government-funded services for seniors living at home. CCACs are responsible for deciding who receives care, the level of care each patient needs and for how long. CCACs do not arrange care through a private company. An individual contacts their local CCAC and is assigned a case manager, who will determine if a patient qualifies for CCAC-funded services. If the person does not qualify, they can arrange and pay for services through a private company. Government-funded services are delivered by health professionals and personal support workers who are under contract with each CCAC. If the individual qualifies for government-funded care, the CCAC will coordinate the application and select the provider. To arrange for private care, the individual must apply directly to the service provider.

PatientServiceIds also provide some control over individuals accessing a patient's record as each individual must input a PatientServiceId, which identifies their approved activities (indicating the reason for access), for a specific patient, and who is paying for this activity. In one embodiment, PatientServiceIds are used to identify the role/organization of the individual who is opening the patient record in addition to the time and duration of each session in the Entity History Index. PatientServiceIds can be used to facilitate cost attribution processes, conducting analytics on the distribution and delivery of health care resources, etc.

In one embodiment, the system uses it's own codes, referred to as PatientServiceIds to track permissions. Look-up tables can be used to correlate PatientServiceIds with External Codes, which are provided by third party organizations such as payors, agencies, etc. (e.g., BRNs, POs, Service Orders etc.) This allows the PatientServiceIds to be associated with third party billing codes. For the purposes of this description, the term BRN will be used to denote the pairing of the PatientServiceId used by the system with the biling code provided by the external party, such as a payor and/or a service providing agency. In general, the user will see and use the BRN, while the system uses the PatientServiceIds. In the example where PatientServiceIds are associated with third party BRNs, BRNs can also function as purchase orders.

Each activity associated with the ordered service will then be associated with the BRN (PatientServiceId). This association may be made at any point during the activity—in some instances it may be beneficial to associate the BRN (PatientServiceId) with a visit to a patient by a clinician, a technician and/or an assistant, where a visit (session) includes one or more transactions. Any transactions under the visit would then inherit the BRN (PatientServiceId) from the visit. In one embodiment, once the visiting clinician, technician and/or assistant arrives at the patient location, they would need to enter the correct BRN (PatientServiceId) into the mobile device in order to open the patient record and gain access to the system.

PatientServiceIds provide several benefits. Since PatientServiceIds are directly or indirectly associated with patient visit activities, and since PatientServiceIds may be associated with BRNs, it allows an administrator to quickly determine who is providing the service/activity and who is responsible for paying for the activity performed on the patient.

One example demonstrating how PatientServiceIds (e.g., BRN) are deployed is presented with reference to FIG. 23 and pertains to a nurse arranging for a technician to visit a patient with Chronic Obstructive Pulmonary Disease (COPD). The partial UI represents a form a Nurse may need to complete prior to accessing a Patient Record. In this example, when the Nurse selects “direct” (as in direct a technician to perform one or more activities during a “visit”) the Directing Nurse will also be required to select an associated BRN that corresponds to the activity. This BRN would then be associated with the one or more activities tied to the “Visit” (or a specific activity as the case may be) as the technician enters data into the technician form. That is, each “Visit” (or specific activity) will be ultimately associated with a BRN. This can help with accounting, invoice generation, or ensuring that the activity has been financially approved, for example.

In particular, FIG. 23 presents an example dashboard view of a nurse, e.g., Mary-Lynn Jameson, R.N. In order to access the patient record on the system, e.g., for Gordon White, the system prompts the nurse to enter information as to what is her reason for accessing the patient record with, “What are you about to do?” In this example, there are three categories the nurse can choose from: Delegate, Chart or Manage, each with specific actions provided to choose from. In this example, the nurse selects to direct a technician visiting with a patient and indicates this by selecting the Direct option.

In this example, under the heading, “How the work is tracked?” the system also prompts the nurse to choose between two possible payors: the SW-CCAC or the VON-Private Pay. In this example, the nurse chooses to select the SW-CCAC option, by selecting the option titled “Directed Tech Visit” in this field, which is labeled Service #003. This field shows the BRN number (2345678-001), the start date (Jan. 12, 2016) and the program (Home First COPD). Therefore, when the technician visits the patient under the direction of the Directing Nurse (Mary-Lynn Jameson RN), the time of the visit will be attributed to BRN 2345678-001. In this example, the Field pertaining to Service #002 indicates that the VON-Private Pay is not to start until Mar. 10, 2016.

In one embodiment, the nurses' time directing a technician also be attributed to a BRN, measured by the start time and stop time of the nurse having the patient record open. The time of an Observing Clinician having a patient record open will also be tracked. In one embodiment, only the visiting time is reported since that would be the time that the care was actually being delivered. In one example, a billing rate would includes both DRN and Tech, for the time that the Tech arrived at the patient's house and/or opened the patient record after arriving at the patient's house.

In other examples a Supervising Therapist may be required to provide a BRN to order a visit by their assistant to provide therapy to a remote patient. For instance, a physiotherapist may be asked, when accessing the patient record to enter data related to the action, to associate the action to a BRN. This BRN may be associated with, by way of non-limiting examples, a pre-authorized therapy plan, or the physiotherapist's billing code.

By way of a non-limiting example, consider therapeutic services. When a Supervising Physician or Nurse orders therapy for a patient, the Supervising Physician or Nurse associates the therapy with a BRN when accessing a Patient Record in the system. Since therapy may involve more than one therapy session, each therapy “action” may be automatically associated with the PatientServiceId for convenience. Alternately the Supervising Physician or Nurse may be required to manually associate the therapy “action” with the BRN for each therapy session.

During each therapy session, a therapist is required to input, into the system, data related to the action performed (in this example, the service provided). The BRN will automatically be associated with this data since the “action” was previously associated with the BRN by the Supervising Physician or Nurse.

When the supervising physician or nurse has ordered another form of therapy for the patient, the supervising physician or nurse would then associate a different BRN with the new therapy plan. Any activities performed by a therapist that are related to this therapy plan would then be associated with the different BRN. Thus, the services of different therapists on the same patient can be differentiated for reasons including, but not limited to, billing, accounting, etc.

### PatientServiceId Architecture

There are a number of different individuals in different roles working for different organizations that need to access a patient record through this system. Each and every time a patient record is accessed, for one of a variety of reasons, a PatientServiceId is entered and used to track the reason for the access and in some cases the duration of the access. Examples of when a patient record will be opened can generally fall into one of a plurality of categories of defined reasons. Some exemplary categories are: 1) to generate a Visit Plan (between the Care Giving Agency and the patient), or a Visit Event, such as a consultation;
2) a non-visit record access event relating to generating a Visit Plan (e.g., Intake/Setup, Review (R/O, Records Update, Chart Audit MDT Review, etc.);
3) an Office Visit template, such as a Directed Technologist Shift, a Nursing shift, a Directed Technologist Visit, a Nursing Visit, a Supervised Therapist Assistant Visit; a Therapist Visit; a Directed Nurse Visit with a Consult; or a Nurse Visit with a Consult;
4) a Non-Visit reason related to the Office visit template to access the patient record (e.g., Nursing Records, Therapy Records, Doctor Review, MDT Review, CCAC view);
5) When access is used to create a Patient Visit Plan, which is a combination of Service Type and Visit Type plus billing model (hours or visits) and planned shift/visit length (controls task timing), list of default visit types (e.g., such as Palliative Tech Shift, Palliative Nurse Visit, Palliative Initial Nurse Visit, a COPD Tech Visit, a CHF Tech Visit, etc.);
6) Non-Visit access plans related to generating an Agency Visit Plan (e.g., Palliative—Records, Complex Care—Records, Palliative—Doctor Review)

Agency Visit Plans comprise a Service Type and a Visit Type in addition to a billing model (hours or visits) and planned shift/visits length (controls task timing), list of default visit tasks.

A Service Type pertains to the structure of the care and patient data collection that has been tailored for a category of patient disease/disorder. For example, categories could include: stroke patients, palliative care, COPD, etc. A series of web forms to be presented on the user interface of a mobile device have been specifically prepared for each category of patient disease/disorder. The Care Agency determines which set(s) of web forms are appropriate for each Service Type.

A Visit Type pertains to who will be visiting the patient and for how long (e.g., For example, will the visit be a technician shift under the supervision of a Directing Clinician (e.g., nurse), or a technician visit under the supervision of a Directing Clinician. Exemplary categories of these kinds of Visit Types comprise: Directed Technologist Shift, a Nursing shift, a Directed Technologist Visit, a Nursing Visit, a Supervised Therapist Assistant Visit; a Therapist Visit; a Directed Nurse Visit with a Consult; or a Nurse Visit with a Consult. There are Visit Action Rules associated with a Visit Type, for example, instructions, supervision, location, with discharged (meaning the purchase order/BRN has ended).

Referring now to FIG. 24, a relational diagram of an embodiment system is provided that depicts how a BRN or PatientServiceId might be used in a system. It will be appreciated that some of the relationships in this diagram may be implemented or executed, at least in part, on multiple devices. These devices can include, but are not limited to, servers, mobile devices, directing workstations, third-party workstations, etc. Some of the functions may also require cooperation and communication between the devices on the system. For instance, “visit” information may be input on a mobile device and saved in a datastore on a server.

PatientServiceIds also link patient, with care team, with physician and payor. PatientServiceId, (access code of “visit” to a patient record) is a many-to-one relationship to BRN—there are many reasons to access records that relate to the same BRN. The PatientServiceId (BRN) identifies what they are doing—so the type of work that they're doing on the patient file when they get access to it with the PatientServiceId and then the PatientServiceId knows what BRN the work was done under.

In one embodiment, PatientServiceIds are used by the system to track who has accessed patient records, why and for how long. For example, if a nurse is visiting a patient and decides to obtain a consultation from another clinician or other user of the system (even the payor to see if the patient can qualify for additional services), the system will keep track of when the consultation was conducted, with whom, and for how long. Thus, this information will be captured in the Electronic Community Care Record (ECCR) as part of the chronological documentation of the provision of care to the patient.

In one embodiment, PatientServiceIds are used by the system to manage approved expenditure of health care resources for a patient. EG. See how dashboard requires the DRN to select a PatientServiceId prior to directing a technician in the field with the patient. The dashboard in this example also indicates the period of time for which a BRN can be used and the time period when another payor will be required.

In this example, a Care provider (e.g., Agency Office) and a Payor (e.g. CCAC—Insurer) collaborate with an Office Program to generate Programs (e.g., service templates). Service templates can be tailored to some degree, if necessary for a particular patient. Each Program would have a Service Level assigned to it in addition to a Discharge Code, indicating the approved duration of time, such that the system will automatically keep track of the duration for a particular program that will be paid for by the payor. In some embodiments, the Program will provide means for another organization or payor to pay for more services after the first service is discharged, and/or the patient to be able to pay for continued services.

In general, an organization will set up what kinds of services it will provide to patients with categories of diseases and disorders (e.g., COPD, stroke, palliative, etc.). In some embodiments, this organization will be a public health care funding organization (e.g., CCAC in Ontario Canada) that contracts with various health care providing agencies. In some embodiments, this organization will be a publically funded (e.g., St. Luke's in England) or privately funded hospital (Kaiser Permanente in California, USA). In some embodiments, this organization will be a health care agency, which bills out to public and/or private payors in order to provide care for patients.

For the purposes of this description of the system, the organization responsible designing the specifics of patient care will be referred to as The Office. The office is usually a collaborative effort between the organization(s) responsible for delivering care to the patient and the organization(s) responsible for funding the care. For example, in some jurisdictions, there may be two or more organizations responsible for providing different aspects of care to a patient (e.g., health care, therapy services, and home care services) and one or more organizations willing to fund these care services (e.g., public and private health care payors).

At a high level, FIG. 24, describes the general set-up of the system for the provision of services to patients. The Office Program 910 refers to the part of the system software used directly and indirectly by the organizations constituting The Office to design the various programs they will provide and fund. In some situations, once a payor's service term runs out, the Office may allow for another payor to continue the service, wherein the next payor may be another funding agency and/or the patient themselves. (For example, in California, once Kaiser Permanente funding is exhausted, a patient may have access to Medical or Medicare funds. Or, perhaps a patient has access to funding from Veteran Affairs, which might cover part of expenses that can then be supplemented with MediCal or Medicare).

The Payor in each of these situations will provide a unique BRN, which will be associated with all of the services they have agreed to fund. In one embodiment, the BRN(s) will be stored in the Patient Service file for each patient using the system. The Office will design a number of Programs 914 that they will offer. Each program will be accorded a Service Level 922 and a Discharge Code 920. Each program uses Service Templates to design the components of each program. If they exhaust their funding under one program and want to continue—they would discharge that particular service and would start up another one. The Discharge is for the service not the patient.

Each program has a Service Type 918, pertaining to the details of the kind of patient data the mobile user will be directed to collect, the kinds of instructions, which will be provided, etc. Each Service Type comprises multiple domains. A domain can refer to nursing, physiotherapy, occupational therapy, speech language therapy, psychology etc. A domain can refer to a diagnosis, such as Stroke, CHF, COPD, End of Life Care. Examples of Service Types 918 would be standardized services for stroke patients, or standardized services for palliative care patients.

As described herein, in order for a nurses license to extend to a technician working with a patient in their home, the detail in these forms must meet regulatory requirements of each jurisdiction. The DF Form Office pertains to the aspect of the system (e.g., Forms Editor), which enables a user to create web forms from the templates.

Each Program also has an Office Visit Plan 916, providing information about who will be visiting the patient in their home or other long-term care facility. The Office Visit Plan 916 comprises one or more Visit Types, where one or more individuals in different roles will be using the web forms presented on their mobile devices to collect information about the patient. In one embodiment the Visit Types can comprise: a Directed Technician Visit, a Directed Technician Shift, a Therapist Visit, a Supervised Therapist Assistant Visit, a Pre-Visit, a Post-Visit. There are also “visits” to the patient record, not the patient, which are included within Visit Types such as Record Updates and Chart Audits. The Role 936, the Visit Action Rule 938, (comprising instructions, supervision, location, with discharged) and Visit Action 932 will be defined for each type of a visit.

The allocation of a specific Patient Service 904 for a specific Patient 900, will be defined according to a Program. The Patient Service 904 record will indicate the BRN number or other billing codes for the Patient Services, the dates and the discharge codes, etc. Each Patient Service 904 will also optionally document one or more Patient Careplan Goals 906 and one or more Patient Careplan Actions 908. In some embodiments there may be Visit Plan Tasks 926 (system & patient), which is NULL for system tasks. Saving a null in a specified identifier field can indicate that it's created by the system and not defined by a user.

Each specific Patient Service 904 will link to data in the patient's record pertaining to each Visit 928 and each Visit Event 930. Because each Visit and Visit events links to a specific Patient Service 906, which comprises one or more BRN's or other types of billing codes, each Visit and Visit Event (including accessing a patient's record to perform a records update or a chart audit) will have a BRN or other billing code linked to it. If it is a Directed Technician or Supervised Assistant visit, then the Directing Nurse or Supervising Therapist's time would also be accorded the BRN or other billing code linked to that service.

FIG. 24 illustrates an exemplary embodiment of the system's relational database structure that uses PatientServiceIds to manage resource allocation, expenditure, and cost attribution for patient care. The PatientServiceIds (in this example, BRN) automatically link each time care is provided to the patient to an approved program comprising an approved Service Level, duration of the service and when the approved service will be terminated (via a Service Code). Referring to FIG. 24, the way that the data is associated in the database is described. In this embodiment the Patient 900 is associated with a single Agency Office 902. The Agency Office 902 identifies the Agency, which is performing the overall care of the Patient 900. An Agency Office 902 may be associated with one or more Office Programs 910. The Office Program 910 is configured to track, at least in part, the service that will be provided to the Patient 900. The Office Program 910 may also be associated with an Insurer 912. The Insurer 912 contains information related to the Insurer or the party responsible for paying for care. It should be noted that different Office Programs 910 may be associated with a single Insurer 912.

The Patient 900 may also be associated with one or more Patient Services 904. The Patient Service 904 stores data related to the service, status, or treatment program that the Patient will be, has, or is currently receiving. In this embodiment the Patient Service can be associated with one or more Patient Careplan Goals 906 and/or Patient Careplan Actions 908. A Patient Careplan Goal 906 includes checkpoint or goal information related to the Patient Service 904. The Patient Careplan Action 908 includes action and/or workplan information related to the Patient Service 904.

The Patient Service 904 may also track billing information associated with the patient. This billing information is typically associated with a single Insurer 912. This billing information includes, but is not limited to, a BRN. Since data related to the service, status, or treatment program is associated with the Patient Service 904, and since the Patient Service 904 also tracks billing information, the actions performed on the Patient 900 can be associated with a BRN either directly or indirectly. Associating a BRN with every billable action performed on a patient may, among other things, simplify accounting, billing, reporting, and invoicing.

In this embodiment the Office Program 910 and the Patient Service 904 are associated with a Program 914. The Program 914 defines, at least in part, the treatment plan that will be provided to a Patient 900. This can include, but is not limited to, Office Visit Plan information 916 and Service Type information 918.

The Program 914 may also be associated with one or more Discharge Codes 920 and/or Service Levels 922. A Discharge Code 920 contains information regarding the completion of a Program 914. For instance, Discharge Codes 920 may track how a Patient 904 completed the Program 914 (e.g., completed, deceased, voluntarily withdrew, dismissed, etc.). Service Level 922 contains information regarding (?).

The Service Type 918 contains information regarding (and informs various aspects of the system of) the type of service that should be provided to the Patient. This can include, but is not limited to, indicating which forms (whether dynamic or static) should be presented to the PSW during site visits. For instance, an example Service Type 918 might be “Stroke” care for stroke victims. The Dynamic Forms (DF) subsystem 924 (or static forms system) will then generate (or present) forms that are directed towards stroke care.

The Program 914 may also be associated with one or more Office Visit Plans 916. The Office Visit Plan 916 may track and/or store, at least in part, office visit instructions and information associated to the Program 914. The Office Visit Plan 916 may also be associated with an Agency Office 902 that is providing/supporting/etc. the office visit.

The Office Visit Plan 916 is associated with one or more Visit Plan Tasks 926. The Visit Plan Tasks 926 track, store, and describe the one or more tasks that should be performed during the visit. The Visit Plan Tasks 926 may also be used to inform the Forms system (e.g., the Dynamic Forms system) so that the appropriate forms may be generated and/or displayed to the technician when the technician enters data related to the visit into the system.

In this embodiment the Patient Service 904 may have one or more Visits 928. A Visit is configured to track, store, and describe, at least in part, information related to each time a therapist/PSW/etc visits a patient. This Visit 928 information is also associated with an Office Visit Plan 916, which allows the specific Visit information to be associated with a Patient treatment plan.

In this embodiment a Visit 928 is associated with one or more Visit Events 930. A Visit Event 930 may be used to outline, describe, or annotate a Visit Action 932. This can include, but is not limited to, to-do lists, procedure lists, patient alerts, etc.

A Visit Action 932 is used to track PSW/therapist activity that is performed in the course of the patient visit. The Visit Action 932 may also be used to store data specific to the action performed. For instance, if a therapist is required, in a workflow, to take a blood pressure of the patient, this information would be stored in the Visit Action 932.

The types of Visit Actions available to the Therapist/PSW are informed by the Visit Type 934, Role 936, and Visit Action Rule 938. The Visit Type 934 is associated with the Office Visit Plan 916, and would store information, procedures, etc. specific to a particular patient visit. The Visit Type is also associated with one or more Visit Action Rules 938. These Visit Action Rules 938 provide controls, at least in part, to the actions that can be taken by a PSW/Therapist. For instance, if the Visit Type 934 indicates that the visit is for monitoring only, the Visit Action Rules 938 would prevent the PSW/Therapist from accessing or modifying patient data unrelated to patient monitoring (e.g., the PSW would not be able to access the Patient Treatment History, etc). The Visit Action Rule 938 is also associated with a Role 936. The Role 936 would also help to control, at least in part, the actions that can be taken by a PSW/Therapist. For instance, if the Role of the person visiting is a Therapist, then the Role 936 would help to prevent non-Therapist information and forms from being presented to the Therapist.

It will be appreciated data from related tables can be searched, reported upon, and edited. For instance, since Visits 928 are associated with Patient Services 924, a report can be generated that associates and displays (to an administrator for example) a BRN (from Patient Services 904) to a Visit 928. Visit Action 932 information and Visit Event 930 information may also be associated with a BRN from the Patient Service 904.

### Forms, Web Pages and Form Templates

The system comprises a number of “forms,” which are used to direct the tech/assistant or other mobile user to perform services for the patient and/or to collect patient data. These forms also include unique identifiers (in the metadata?) such that each time they are used, this fact is recorded in the Entity History Index and each time they are sent, notifications are sent to the appropriate dashboard, mobile device or workstation of another user of the system.

In the medical field, the specifics of a form, reflecting the directed data collection workflow is critical, in fact the legal transference of a nurse's license (who could be working out of his/her home) to the technician working in a patient's home, for example, depends upon the specific content and the process of documentation in the patient's record. Thus, not only the processes of delivering appropriate specific content (i.e., specific data collection workflow UIs) in a timely manner to the technician, the responses delivered to the directing nurse, the notifications of the communications, the data storage, etc. require specific metadata to be processed.

There are two kinds of forms—template-based forms that can be defined through a forms builder user interface (Dynamic Forms) and those which are hard-coded and more traditionally developed. These forms are web pages presented on the workstations and mobile device's of the users. Both types of forms also include unique identifiers in the metadata (EntityTypeId) such that each time they are used, this fact is recorded in the Entity History Index Table 100 along with the EntityId for each instance of a form's use (e.g., see FIG. 8).

Hard coded forms are used when the type and/or structure of the information being collected is specialized, structured and is a basic requirement of the medical system. Some non-limiting examples of hard coded forms are used to collect: patient information (i.e., name, home address, phone number, birthdate, etc.); medications; Nurse Clinical Notes; Tech/Assistant Clinical Notes (largely text boxes); and instructions. Some tables corresponding to hard-coded forms are illustrated in FIG. 8: Visit Event Table 114, Clinical Note Table 128, Medication Change Table 129, Medication Given Table 132, Medication Table 130, Instruction Table 134, etc.

The template forms are employed when the details of a form vary significantly and need to be customized for each service (e.g., patient vital signs, mood, physiological indicators, etc.). Form templates avoid the need of having to write new code for each form generated for use within the system. As understood by one skilled in the art, templates are structured in standardized patterns, wherein the creator of a specific form only needs to define the attributes of a field, rather than write new code. Templates can be designed as forms used to collect a wide variety of patient data such as a form used to collect a patient's vital state readings such as heart rate, blood pressure, temperature, or a form used to collect information regarding a patient's psychological status, etc. Once the code for the form is standardized into structural patterns amenable to a template structure, then the template just needs to be provided with definitions of the kinds of data to be encompassed within the form. FIG. 8 illustrates exemplary tables corresponding to Dynamic Forms, the Dynamic Form Table 126 and the Dynamic Form Data Table 127.

One skilled in the art would appreciate that there are a variety of types of template forms that could be used with this system for example: Cardiovascular, Eye/Ear/Nose & Throat, Gastrointestinal, Genitourinary, Neurology, Respiratory, Skin Integrity, Vital Signs, etc.

### Form Versioning

The form metadata in the Dynamic Forms also enables the chronological reporting of what form was used when, and in the case when a specific form (e.g., patient vital signs) changes over time, the system will display the data in the original form template version that was used to collect the data. This is possible because of Form Versioning.

In one embodiment, the system is deployed over a large geographical area such as a Province or a State such that multiple primary care agencies (or therapy service providers) could be users of the system. In this embodiment a situation may exist wherein specific primary care agencies or service providers may require their own version of a form to be used. If a patient is cared for in one region and then transferred to another region where they will be provided care by different agencies or service providers and different forms might be used, the system will keep track of exactly which Dynamic Form was used, when and by whom. A report of the Clinical History will reflect data in the original forms that were used to collect the data.

In some example embodiments the form service 700 may incorporate metadata associated with the form template. For instance, in some embodiments the form service 700 may store version information associated with the form template. Newly generated form templates may start with a base version number, and once new versions of the form are deployed the new version of the form may be issued a version number that is different from the base version number. Furthermore, the metadata may also include data associated with a workflow traceability. For instance, in-progress form template edits may be tagged with a metadata indicating the in-progress status of the form template. Alternate tags, such as approved, archived, published, etc., can be used to associate the form template with the appropriate workflow status.

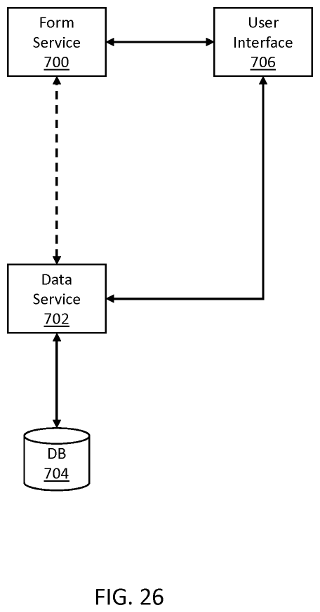

Referring now to FIG. 26, a block diagram of an example embodiment is depicted. In this embodiment the dynamic form system includes a form service 700, a data service 702, a database 704, and a user interface 706. The form service 700 is configured to provide form templates (including reusable UI forms, layout, validation rules, revision data, and behavior) to the user interface 706. The data service 702 is configured to provide data for use in the form templates to the user interface 706. The data service 702 stores and retrieves data from a database 704. The form service 700 and data service 702 are communicatively connected so that instructions can be communicated directly between the form service 700 and the data service 702.

The user interface 706 is configured to allow a user to view, input, and edit data. The data, as retrieved from the data service 702, is presented to the user via the form template 700. The user may then input or edit data via the data service 702.

It will be appreciated that the form service 700, data service 702, database, 704, and user interface 706 may be implemented on one or more computing devices. In an example embodiment, the form service 700, data service 702, user interface 706, and database 704 may be a part of a web application implemented on a server. In another example embodiment, each of the form service 700, data service 702, user interface 706, and database 704 may be components implemented in a cloud computing environment such as MICROSOFT AZURE, AMAZON EC2, or GOOGLE CLOUD SERVICES.

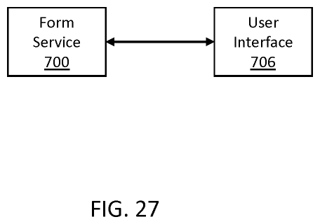

Referring now to FIG. 27, a block diagram of an example embodiment for creating form templates (including reusable UI forms, layout, and behavior) is provided. In this example the user interface 706 is configured to communicate directly with the form service 700 so that a user may define form templates (including reusable UI forms, layout, and behavior). In this example embodiment the user has no direct access to the data service 702. Once the user has completed defining a new form template (including reusable UI forms, layout, and behavior), the form template may be saved in a data store (not shown) of the form service 700. In another example embodiment, the form service 700 may be communicatively connected to a database 704, which may act as a data store for the form service 700.

In some example embodiments the form service 700 may incorporate metadata associated with the form template. For instance, in some embodiments the form service 700 may store version information associated with the form template. Newly generated form templates may start with a base version number, and once new versions of the form are deployed the new version of the form may be issued a version number that is different from the base version number. Furthermore, the metadata may also include data associated with a workflow traceability. For instance, in-progress form template edits may be tagged with a metadata indicating the in-progress status of the form template. Alternate tags, such as approved, archived, published, etc., can be used to associate the form template with the appropriate workflow status.

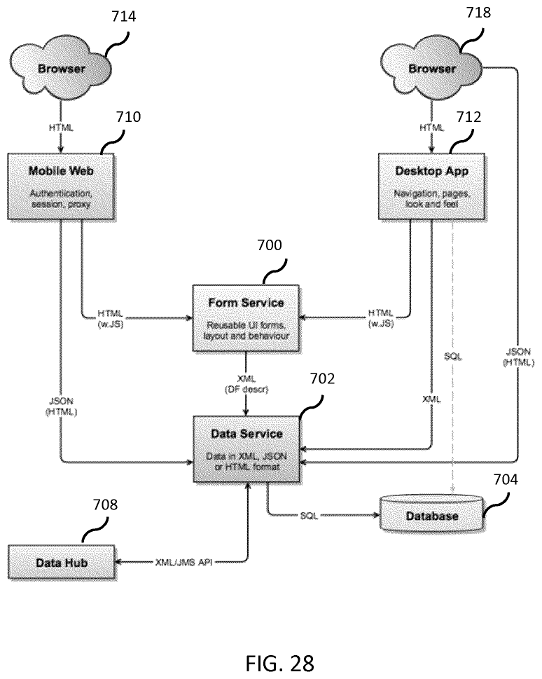

Referring now to FIG. 28, a block diagram of an example embodiment is depicted. This example illustrates the dynamic form system and the underlying data formats/markup languages used in an example dynamic form system. In this example, the form service 700 is configured to provide form template data to a mobile web browser 714 associated with a mobile web app 712, a desktop browser 718 associated with a desktop app 712, or both, in HyperText Markup Language (HTML) and/or JavaScript (JS). The data service 702 is configured to transmit and receive data (including patient data and/or data originating from the database 704 or data hub 708) to and from the form service 700, a mobile web browser associated with a mobile web app, a desktop browser associated with a desktop app, or any combination of the three, using eXtensible Markup Language (XML), JavaScript Object Notation (JSON), or both. The data service 702 is further configured to communicate with the database 704 using Sequential Query Language (SQL). The data service 702 is further configured to communicate with the data hub 708 using an Application Program Interface (API) that accepts data in either the XML format or the Java Message Service (JMS) format.

Referring now to FIG. 716, a block diagram of an example workflow and data flow diagram for an input/output dynamic form template is provided. In this example an application (e.g., a mobile web application or desktop app), via an app wrapper 716, requests a form template 720 from the form service 700. The form service 700 is configured to retrieve the appropriate form template 300 and send it to the browser (using HTML, JS, or both) so that the form template 720 may be rendered.

In this example the form template 720 includes one or more fields for data display and/or entry. In this example some fields (such as the patient's name) may be read-only. Other fields may be marked read-write.

In this example the form data fields 722 in the form template 720 correspond to data stored in the database 704. However, the form template 720 does not contain a direct link to the data in the database 704. Instead the form template 720 contains a reference to a corresponding data entry, data view, and/or data table in the database 704. This reference is defined in the data service 702 and is used by the form template 720 to link, indirectly, the form data field 722 to the corresponding data entry, data view, and/or data table in the database 704. This effectively allows the form data field 722 to be abstracted away from the particulars of the database 704. This can allow for, amongst other things, reusability (previously defined form data field 722 references can be reused), abstraction (the query details to access/find data in the database can be hidden from the form template designer—i.e., the information is encapsulated in the reference on the data service 702), etc.

The reference may also include instructions (in this case, JS with the jQuery third-party library) to retrieve data from the data service 702 and render the relevant data in the browser. This data may correspond to patient data stored in the database 704. This data may also be retrieved synchronously or asynchronously (using AJAX requests) relative to the form template 720 request.

Once the user enters data into the form template 720 and submits (or saves) the form, the data in the form fields 722 is sent to the data service 702 for processing. In this example, the data is first processed using Knockout software (KO). KO is a third-party view/viewmodel framework that assists in the translation of the data entered in the forms to JSON. This JSON data is then sent to the data service 702 for saving in the database 704, effectively separating the view (i.e., form template) from the data.

In some examples it may be appropriate to pre-fill form fields (or the fields in form sets) and allow the user to edit these form fields from the browser. In this case the data service 702 may also work with KO to pre-populate the form template with data from the database 704.

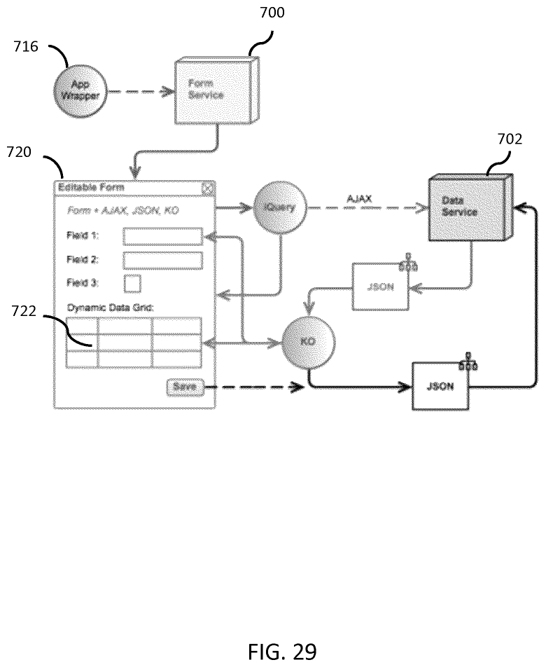

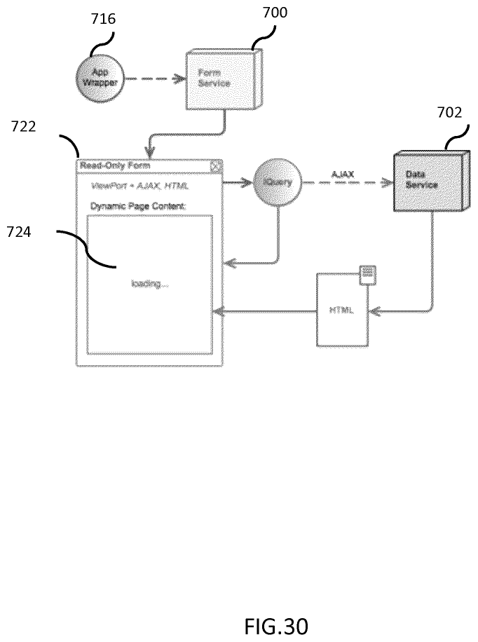

Referring now to FIG. 30, a block diagram of an example workflow and data flow diagram for a read-only dynamic form template is provided. The workflow is similar to that of FIG. 29. However, since the form template is read-only (i.e., is not configured to accept input), the data retrieved by the data service 702 can be formatted in HTML and displayed in the browser directly.

The following clauses are offered as further description of the examples of the apparatus. Any one or more of the following clauses may be combinable with any another one or more of the following clauses. Any one of the following clauses may stand on its own merit without having to be combined with another other of the above-identified clauses. Clause (1): A system comprising: a server having a database having an electronic community care record (ECCR); a directing workstation having a user interface for allowing a licensed healthcare professional to access the ECCR; a mobile device having a user interface for allowing a healthcare assistant to access the ECCR; the ECCR comprising an entity history index that contains data corresponding to an action performed on the user interface of the directing workstation and the mobile device. Clause (2) The system of any one or a combination of clauses in this paragraph wherein instructions for treatment data are included in the ECCR. Clause (3): The system of any one or a combination of clauses in this paragraph wherein the instructions for treatment data include a workflow having a workflow state and a workflow status. Clause (4): The system of any one or a combination of clauses in this paragraph wherein the user interface of the directing workstation, mobile device, or both will change depending on the workflow. Clause (5): The system of any one or a combination of clauses in this paragraph wherein an alert is sent to the user interface of the mobile device, the directing workstation, or both once a specific workflow state has been reached. Clause (6) The system of any one or a combination of clauses in this paragraph wherein the healthcare assistant can access instructions for treatment from the ECCR. Clause (7) The system of any one or a combination of clauses in this paragraph further comprising a supervisory mode for supervising the healthcare professional on the directing workstation wherein a supervisor (observing clinician) on a second directing workstation can monitor the activity on the directing workstation (directing clinician), the activity of the mobile device, or both. Clause (8) The system of any one or a combination of clauses in this paragraph further comprising an instruction mode for issuing instructions to a healthcare assistant wherein the licensed healthcare professional can instruct, in real time or near real time, the healthcare assistant, and a data generated by the instruction mode is stored in the ECCR. Clause (9) The system of any one or a combination of clauses in this paragraph further comprising a collaboration mode for developing, at least in part, a treatment plan wherein one or more healthcare workers, each on their own directing workstation, can communicate (remotely monitor) in real (or near real) time with one or more healthcare assistants, each on their own mobile device. Clause (10) The system of any one or a combination of clauses in this paragraph wherein the entity history index data includes timestamp data, user data, and data on whether an attribute of the ECCR was created, reversed, updated, or deleted. Clause (11) The system of any one or a combination of clauses in this paragraph wherein the ECCR includes entity data that includes any combination of patient data, user access data, workflow data, metadata, billing data, and history data. Clause (12) The system of any one or a combination of clauses in this paragraph further comprising a dynamic forms system for generating forms that are displayed on the directing workstation, the mobile device, or both. Clause (13) The system of any one or a combination of clauses in this paragraph wherein the dynamic forms system further includes a versioning system for tracking versions of the form as the form is changed. Clause (14) The system of any one or a combination of clauses in this paragraph wherein the dynamic forms system generates forms based on a workflow defined in the ECCR. Clause (15) The system of any one or a combination of clauses in this paragraph wherein a cost attribution record is included in the ECCR. Clause (16) The system of any one or a combination of clauses in this paragraph further comprising a views system for generating views of ECCR information on the directing workstation, the mobile device, or both. Clause (17) The system of any one or a combination of clauses in this paragraph wherein the views system generates views based on rendering tables defined on the server.

This written description uses examples to disclose the invention, including the best mode, and also to enable any person skilled in the art to make and use the invention. The patentable scope of the invention is defined by the claims, and may include other examples that occur to those skilled in the art. Such other examples are within the scope of the claims if they have structural elements that do not differ from the literal language of the claims, or if they include equivalent structural elements with insubstantial differences from the literal language of the claims.

It may be appreciated that the assemblies and modules described above may be connected with each other as required to perform desired functions and tasks within the scope of persons of skill in the art to make such combinations and permutations without having to describe each and every one in explicit terms. There is no particular assembly or component that may be superior to any of the equivalents available to the person skilled in the art. There is no particular mode of practicing the disclosed subject matter that is superior to others, so long as the functions may be performed. It is believed that all the crucial aspects of the disclosed subject matter have been provided in this document. It is understood, for this document, that the phrase “includes” is equivalent to the word “comprising.” The foregoing has outlined the non-limiting embodiments (examples). The description is made for particular non-limiting embodiments (examples). It is understood that the non-limiting embodiments are merely illustrative as examples.

What is claimed is:

1. A system comprising:
a server having a database having an electronic community care record (ECCR);
a directing workstation having a user interface for allowing a licensed healthcare professional to access the ECCR;
a mobile device having a user interface for allowing a healthcare assistant to access the ECCR;
the ECCR comprising an entity history index that contains data corresponding to an action performed on the user interface of the directing workstation and the mobile device.

2. The system of claim 1 wherein instructions for treatment data are included in the ECCR.

3. The system of claim 2 wherein the instructions for treatment data include a workflow having a workflow state and a workflow status.

4. The system of claim 3 wherein the user interface of the directing workstation, mobile device, or both will change depending on the workflow.

5. The system of claim 3 wherein an alert is sent to the user interface of the mobile device, the directing workstation, or both once a specific workflow state has been reached.

6. The system of claim 1 wherein the healthcare assistant can access instructions for treatment from the ECCR.

7. The system of claim 1 further comprising a supervisory mode for supervising the healthcare professional on the directing workstation wherein a supervisor (observing clinician) on a second directing workstation can monitor the activity on the directing workstation (directing clinician), the activity of the mobile device, or both.

8. The system of claim 1 further comprising an instruction mode for issuing instructions to a healthcare assistant wherein the licensed healthcare professional can instruct, in real time or near real time, the healthcare assistant, and a data generated by the instruction mode is stored in the ECCR.

9. The system of claim 1 further comprising a collaboration mode for developing, at least in part, a treatment plan wherein one or more healthcare workers, each on their own directing workstation, can communicate (remotely monitor) in real (or near real) time with one or more healthcare assistants, each on their own mobile device.

10. The system of claim 1 wherein the entity history index data includes timestamp data, user data, and data on whether an attribute of the ECCR was created, reversed, updated, or deleted.

11. The system of claim 1, wherein the ECCR includes entity data that includes any combination of patient data, user access data, workflow data, metadata, billing data, and history data.

12. The system of claim 1 further comprising a dynamic forms system for generating forms that are displayed on the directing workstation, the mobile device, or both.

13. The system of claim 12 wherein the dynamic forms system further includes a versioning system for tracking versions of the form as the form is changed.

14. The system of claim 12 wherein the dynamic forms system generates forms based on a workflow defined in the ECCR.

15. The system of claim 1 wherein a cost attribution record is included in the ECCR.

16. The system of claim 1 further comprising a views system for generating views of ECCR information on the directing workstation, the mobile device, or both.

17. The system of claim 16 wherein the views system generates views based on rendering tables defined on the server.
